(19) United States
(12) Patent Application Publication
(10) Pub. No.: US 2020/0405643 A1
(43) Pub. Date: Dec. 31, 2020

(54) DRUG COMPOSITIONS CONTAINING POROUS CARRIERS MADE BY THERMAL OR FUSION-BASED PROCESSES

(71) Applicant: BOARD OF REGENTS, THE UNIVERSITY OF TEXAS SYSTEM, Austin, TX (US)

(72) Inventors: Robert O. WILLIAMS, III, Austin, TX (US); Masataka HANADA, Austin, TX (US); Scott V. JERMAIN, Austin, TX (US)

(21) Appl. No.: 16/646,461

(22) PCT Filed: Sep. 11, 2018
(86) PCT No.: PCT/US18/50323

§ 371 Date: Mar. 11, 2020

Related U.S. Application Data
(60) Provisional application No. 62/556,954, filed on Sep. 11, 2017.

(51) Int. Cl. A61K 9/14; A61K 9/16

(52) U.S. Cl. CPC A61K 9/146; A61K 45/06; A61K 9/143; A61K 9/1682

(57) ABSTRACT

Pharmaceutical compositions for improving the solubility and dissolution of poorly soluble drugs, which contain a therapeutic agent, a pharmaceutically acceptable polymer, and a mesoporous carrier. These pharmaceutical compositions have been prepared by thermal processes and fusion-based high energy mixing processes that do not require external heat input to obtain a composition that shows improved properties. Also provided herein are methods of preparing and use thereof

This application is a national phase application under 35 U.S.C. § 371 of International Application No. PCT/US2018/050323, filed Sep. 11, 2018, which claims the benefit of U.S. Provisional Application No. 62/556,954, filed on Sep. 11, 2017, the entire contents of each of which is hereby incorporated by reference.

### BACKGROUND

### 1. Field

The present disclosure relates generally to the field of pharmaceuticals and pharmaceutical manufacture. More particularly, it concerns compositions and methods of preparing a drug composition containing mesoporous carriers, therapeutic agents, and pharmaceutically acceptable polymers which have been processed through thermal or fusion-based processes (i.e. fusion-based high energy mixing processes that do not require external heat input.

### 2. Description of Related Art

Amorphous solid dispersions (ASDs) have been shown to enhance the solubility and dissolution rate of BCS II and IV drugs by employing a “spring and parachute” effect (Brouwers et al., 2009). The “spring and parachute” effect occurs as the ASD is placed in the desired dissolution media, quickly springing the drug into a supersaturated solubility state and employing a polymeric stabilizer to function as a parachute in an attempt to inhibit recrystallization and an unwanted return to equilibrium solubility (Brough and Williams, 2013; Brouwers et al., 2009; Guzmán et al., 2007). Inhibiting recrystallization and a subsequent return to equilibrium solubility allows maintenance of elevated drug concentration during its intestinal transit, in turn allowing for the maximum systemic exposure. In order to obtain this effect, ASDs typically necessitate a high polymer ratio compared to the active compound to maintain amorphicity (Brough and Williams, 2013). The higher polymer ratios necessary for the “spring and parachute” effect tend to decrease disintegration time and drug release into the test media due to polymer viscosity when formulated as tablets (Goddeeris et al., 2008). Furthermore, polymer selection is a critical aspect to form stable ASD formulations and can affect the amount of drug that is loaded and its ability to maintain amorphicity.

Previous reports include preparation methods of ASDs composed of porous carriers for the purpose of improving dissolution and stability (Bhargavi et al., 2015; Hoashi et al., 2011; Lainé et al., 2016; Maniruzzaman et al., 2015; Münzenberg and Moller, 2017; Nakahashi et al., 2014; Patel and Dave, 2013; Shibata et al., 2009; Takeuchi et al., 2005; Van Speybroeck et al., 2010; Hong et al., 2016; Watanabe et al., 2002; Bahl and Bogner, 2006; Fujii et al., 2005). The use of mesoporous silica (MPS), an example of a porous carrier, as a pharmaceutical excipient has been primarily reported to improve flowability with little emphasis on its ability to improve dissolution or show beneficial dissolution properties. The adsorption of drug onto the silica particles is achieved via an immersion method (Bhargavi et al., 2015; Münzenberg and Moller, 2017), evaporation method (Lainé et al., 2016), and spray drying method (Takeuchi et al., 2005; Hong et al., 2016). However, in each of these aforementioned techniques, it is necessary to first dissolve the drug in an organic solvent. The necessity of drug loading using solvent imparts high variability on the average achievable drug loading (13.2-53.4% w/w), which is dependent on such factors as the specific drug, physicochemical properties of the drug, solvent type, and solvent properties (Salonen, et al., 2005) and thus limiting the usefulness of this technique. Others have sought to avoid the use of solvents altogether and reported formulations containing MPS prepared by applying heat and mechanical shear using a ball mill (Nakahashi et al., 2014; Watanabe et al., 2002), a high-speed elliptical-rotor type blender (Fujii et al., 2005), a twin-screw extruder (Maniruzzaman et al., 2015; Shibata et al., 2009), and a twin-screw kneader (Hoashi et al., 2011; Shibata et al., 2009). In addition, it has been reported that porous carriers (other than MPS) were prepared using magnesium aluminometasilicate (Maniruzzaman et al., 2015) and crospovidone (Shibata et al., 2009; Fujii et al., 2005). However, each of these processes has limitations that can render a formulation unsuccessful. For hot melt extrusion, using an extruder or kneader is associated with increased risk of drug degradation when thermolabile drugs are processed above their melting point (DiNunzio et al., 2010). In addition, when dry milling, there is a risk of metal contamination for pulverization using media such as balls and beads, and it is necessary to apply mechanical stress for prolonged periods of time (0.5-3 hours) to induce amorphicity of crystalline drugs (Watanabe et al., 2003; Watanabe et al., 2001).

Thus, there is still a need develop a high drug loaded formulation (≥50% w/w) that exhibits high and sustained drug dissolution, similar the “spring and parachute” effect.

### SUMMARY OF THE INVENTION

In some aspects, the present disclosure provides pharmaceutical composition containing one or more therapeutic agents, a mesoporous carrier, and one or more pharmaceutically acceptable polymers, which have been processed through a thermal or fusion-based high energy process and exhibit improved properties such as dissolution and solubility.

In still another aspect, the present disclosure provides pharmaceutical composition comprising:
(A) a therapeutic agent, wherein the therapeutic agent comprises at least about 50% w/w of the pharmaceutical composition;
(B) one or more pharmaceutically acceptable polymers; and
(C) a non-preloaded mesoporous carrier.

In some embodiments, the pharmaceutical composition is prepared using a thermal process or a fusion-based high energy mixing process that does not require external heat input. In one embodiments, the thermal process is hot melt extrusion. Alternatively, the thermal process may be a hot melt granulation process. In some embodiments, the thermal process is carried out at a temperature below the melting point of the therapeutic agent. Additionally, the thermal process may be carried out at a temperature below the decomposition temperature of the therapeutic agent as measured by thermogravimetric analysis. In other embodiments, the composition is processed through a fusion-based high energy mixing process that does not require external heat input that results in an increase in temperature such as an increase in temperature that results from frictional or shear energy. In some embodiments, the composition has been processed by a thermokinetic mixing process. In some embodiments, the components have not been milled prior to the hot melt process. In one embodiment, the pharmaceutical composition is substantially free of a solvent. Furthermore, the pharmaceutical composition may be essentially free of a solvent. In some embodiments, the pharmaceutical composition has been processed without the addition of a solvent. Additionally, the pharmaceutical composition may have been prepared without the addition of a solvent.

In some embodiments, the therapeutic agent has a solubility in water of less than about 5 mg/mL including therapeutic agents which are a Biopharmaceutics Classification System Class II or IV compound. Additionally, the therapeutic agent may also be known to undergo thermal degradation. In one embodiment, the pharmaceutically acceptable polymer is a cellulosic polymer such as a neutral cellulosic polymer or an ionizable cellulosic polymer. In another embodiment, the pharmaceutically acceptable polymer is a non-cellulosic polymer such as a neutral non-cellulosic polymer or an ionizable non-cellulosic polymer. In one embodiment, the pharmaceutically acceptable polymer is a polymethacrylate or polyacrylate functionalized with a carboxylic acid group.

In some embodiments, the mesoporous carrier is a silica carrier, an alumina carrier, a mixed alumino-silicate carrier, a mixed inorganic oxide carrier, a calcium carbonate carrier, or a clay carrier. In one embodiment, the mesoporous carrier is a mesoporous silica or silicate. In some embodiments, the mesoporous carrier is a mesoporous silica such as a hydrous silicon dioxide, a mesoporous fumed silica, or a mesoporous magnesium aluminum silicate. In one embodiment, the mesoporous carrier has not been loaded with the therapeutic agent before the formulation with the pharmaceutically acceptable polymer. Furthermore, the mesoporous carrier may not have been loaded with any therapeutic agent prior to formulation with the therapeutic agent and the pharmaceutically acceptable polymer.

In one embodiment, the pharmaceutically acceptable polymer and the therapeutic agent form a mixture having a Flory-Huggins interaction parameter (χ) of greater than 0.25 as determined by differential scanning calorimetry (DSC) such as greater than 1. Furthermore, the pharmaceutically acceptable polymer and the therapeutic agent may form a mixture having a positive ΔGₘᵢₓ as determined by DSC. In some embodiments, the pharmaceutical composition has a specific surface area of greater than about 5 m²/g as measured by BET, greater than about 10 m²/g, greater than about 15 m²/g, or greater than about 20 m²/g.

In some embodiments, the pharmaceutical composition comprises from about 50% w/w to about 98% w/w therapeutic agent relative to the total weight of the pharmaceutical composition, such as from about 50% w/w to about 75% w/w therapeutic agent relative to the total weight of the pharmaceutical composition or from about 50% w/w to about 60% w/w therapeutic agent relative to the total weight of the pharmaceutical composition. Additionally, the pharmaceutical composition may comprise from about 1% w/w to about 49% w/w mesoporous carrier relative to the total weight of the pharmaceutical composition such as from about 10% w/w to about 40% w/w mesoporous carrier relative to the total weight of the pharmaceutical composition, from about 15% w/w to about 35% w/w mesoporous carrier relative to the total weight of the pharmaceutical composition, or from about 25% w/w to about 35% w/w mesoporous carrier relative to the total weight of the pharmaceutical composition. In some embodiments, the pharmaceutical composition comprises from about 1% w/w to about 49% w/w pharmaceutically acceptable polymer relative to the total weight of the pharmaceutical composition such as from about 10% w/w to about 40% w/w pharmaceutically acceptable polymer relative to the total weight of the pharmaceutical composition, from about 15% w/w to about 35% w/w pharmaceutically acceptable polymer relative to the total weight of the pharmaceutical composition, or from about 15% to about 25% w/w pharmaceutically acceptable polymer relative to the total weight of the pharmaceutical composition.

In one embodiment, the pharmaceutically acceptable polymer is not polyvinyl pyrrolidone (PVP). In some embodiments, the pharmaceutically acceptable polymer is not a neutral non-cellulosic polymer. In one embodiment, the therapeutic agent is not troglitazone. In some embodiments, the therapeutic agent is not a thiazolidinedione.

In some embodiments, the pharmaceutical compositions further comprise an excipient such as a lubricant, disintegrant, binder, filler, surfactant, or any combination thereof.

In yet another aspect, the present disclosure provides solvent free methods of preparing a pharmaceutical composition comprising:

(A) obtaining a composition comprising:
(1) a therapeutic agent, wherein the therapeutic agent comprises at least about 50% w/w of the composition;
(2) a mesoporous carrier; and
(3) one or more pharmaceutically acceptable polymers;
(B) heating the composition through a thermal process or a fusion-based high energy mixing process to form a pharmaceutical composition.

In some embodiments, the composition is obtained by adding the therapeutic agent, the mesoporous carrier, and the pharmaceutically acceptable polymers. Alternatively, the composition may be obtained by admixing the therapeutic agent, the mesoporous carrier, and the pharmaceutically acceptable polymers. In other embodiments, the composition is obtained from a third party.

In one embodiments, the thermal process is hot melt extrusion. Alternatively, the thermal process may be a hot melt granulation process. In some embodiments, the thermal process is carried out at a temperature below the melting point of the therapeutic agent. Additionally, the thermal process may be carried out at a temperature below the decomposition temperature of the therapeutic agent as measured by thermogravimetric analysis. In other embodiments, the composition is processed through a fusion-based high energy mixing process that does not require external heat input that results in an increase in temperature such as an increase in temperature that results from frictional or shear energy. In some embodiments, the composition has been processed by a thermokinetic mixing process.

In one embodiment, the pharmaceutically acceptable polymer and the therapeutic agent form a mixture having a Flory-Huggins interaction parameter (χ) of greater than 0.25 as determined by differential scanning calorimetry (DSC) such as greater than 1. Furthermore, the pharmaceutically acceptable polymer and the therapeutic agent may form a mixture having a positive ΔGₘᵢₓ as determined by DSC. In some embodiments, the composition has a specific surface area of greater than about 5 m²/g as measured by BET, greater than about 10 m²/g, greater than about 15 m²/g, or greater than about 20 m²/g.

In some embodiments, the composition comprises from about 50% w/w to about 98% w/w therapeutic agent relative to the total weight of the composition, such as from about 50% w/w to about 75% w/w therapeutic agent relative to the total weight of the composition or from about 50% w/w to about 60% w/w therapeutic agent relative to the total weight of the composition. Additionally, the composition may comprise from about 1% w/w to about 49% w/w mesoporous carrier relative to the total weight of the composition such as from about 10% w/w to about 40% w/w mesoporous carrier relative to the total weight of the composition, from about 15% w/w to about 35% w/w mesoporous carrier relative to the total weight of the composition, or from about 25% w/w to about 35% w/w mesoporous carrier relative to the total weight of the composition. In some embodiments, the composition comprises from about 1% w/w to about 49% w/w pharmaceutically acceptable polymer relative to the total weight of the composition such as from about 10% w/w to about 40% w/w pharmaceutically acceptable polymer relative to the total weight of the composition, from about 15% w/w to about 35% w/w pharmaceutically acceptable polymer relative to the total weight of the composition, or from about 15% to about 25% w/w pharmaceutically acceptable polymer relative to the total weight of the composition.

In one embodiment, the thermal process is hot melt extrusion and the screw speed of the of the extruder is from about 10 rpm to about 500 rpm such as from about 50 rpm to about 250 rpm. In another embodiment, the thermal process is hot melt granulation process and comprises heating the composition for a time period from about 2 minutes to about 3 hours such as about 5 minutes to about 1 hour. In some embodiments, the methods further comprise milling the pharmaceutical composition. The methods may further comprise sieving the pharmaceutical composition such as sieving through a screen with a pore size from about 100 μm to about 500 μm. In some embodiments, the methods are substantially free of a solvent. Furthermore, the methods may be essentially free of a solvent.

In some embodiments, the therapeutic agent has a solubility in water of less than about 5 mg/mL including therapeutic agents which are a Biopharmaceutics Classification System Class II or IV compound. Additionally, the therapeutic agent may also be known to undergo thermal degradation. In one embodiment, the pharmaceutically acceptable polymer is a cellulosic polymer such as a neutral cellulosic polymer or an ionizable cellulosic polymer. In another embodiment, the pharmaceutically acceptable polymer is a non-cellulosic polymer such as a neutral non-cellulosic polymer or an ionizable non-cellulosic polymer. In one embodiment, the pharmaceutically acceptable polymer is a polymethacrylate or polyacrylate functionalized with a carboxylic acid group.

In some embodiments, the mesoporous carrier is a silica carrier, an alumina carrier, a mixed alumino-silicate carrier, a mixed inorganic oxide carrier, a calcium carbonate carrier, or a clay carrier. In one embodiment, the mesoporous carrier is a mesoporous silica or silicate such as a mesoporous silica, mesoporous fumed silica, or mesoporous magnesium aluminum silicate.

In some embodiments, the compositions further comprise an excipient such as a lubricant, disintegrant, binder, filler, surfactant, or any combination thereof.

In still some aspects, the present disclosure provides pharmaceutical compositions prepared according to the methods described herein. In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crémes, in lipid compositions, via a catheter, via a lavage, via continuous infusion, via infusion, via inhalation, via injection, via local delivery, or via localized perfusion. The pharmaceutical compositions may be formulated for oral administration such as in a hard or soft capsule, a tablet, a syrup, a suspension, an emulsion, a solution, or a wafer.

In still another aspect, the present disclosure provides methods of treating a disease or disorder in a patient in need thereof comprising administering to the patient a therapeutically effective amount of a pharmaceutical composition described herein comprising a therapeutic agent effective to treat the disease or disorder.

In yet another aspect, the present disclosure provides methods of preventing a disease or disorder in a patient in need thereof comprising administering to the patient a therapeutically effective amount of a pharmaceutical composition described herein comprising a therapeutic agent effective to prevent the disease or disorder.

It is contemplated that any embodiment discussed in this specification can be implemented with respect to any method or composition of the invention, and vice versa. Furthermore, compositions of the invention can be used to achieve the methods of the invention.

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate, for example, that a value includes the inherent variation of error for the device, the method being employed to determine the value, or the variation that exists among the study subjects or experimental studies. In the context of this invention, “about” means approximate, and unless otherwise indicated, further means plus/minus 10%.

As used herein, the term “substantially free of” or “substantially free” in terms of a specified component, is used herein to mean that none of the specified component has been purposefully formulated into a composition and/or is present only as a contaminant or in trace amounts. The total amount of all contaminants, by-products, and other material is present in that composition in an amount less than 2%. The term “more substantially free of” or “more substantially free” is used to represent that the composition contains less than 1% of the specific component. The term “essentially free of” or “essentially free” contains less than 0.5% of the specific component.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows thermogravimetric analysis of IND demonstrating onset of degradation at approximately 160° C.

FIG. 2 shows the APPEARANCE of the processed XDP and HM granules.

FIGS. 3A-3D show PXRD of IND:XDP formulations (FIG. 3A); IND:AF15 and IND:AF15:XDP formulations (FIG. 3B); IND:VA64 and IND:VA64:XDP formulations (FIG. 3C); and IND:KIR and IND:KIR:XDP formulations (FIG. 3D).

FIGS. 4A-4D show mDSC of IND:XDP formulations (FIG. 4A); IND:AF15 and IND:AF15:XDP formulations (FIG. 4B); IND:VA64 and IND:VA64:XDP formulations (FIG. 4C); and IND:KIR and IND:KIR:XDP formulations (FIG. 4D).

FIG. 5 shows appearance of HME samples prior to milling and sieving. The green square indicates complete amorphicity (1 scale=1 mm).

FIG. 6 shows mDSC of ternary ASDs prepared by different HME conditions.

FIGS. 7A-7D show dissolution profiles of binary and ternary formulations (n=3) of IND:XDP formulations (FIG. 7A); IND:AF15 and IND:AF15:XDP formulations (FIG. 7B); IND:VA64 and IND:VA64:XDP formulations (FIG. 7C); and IND:KIR and IND:KIR:XDP formulations (FIG. 7D) and compares these results to the dissolution

FIG. 8 shows dissolution profiles (n=3) of ternary ASDs.

FIG. 9 shows complex VISCOSITY of IND:Polymer (5:2) at 150° C. during a time period of 10 minutes.

FIG. 10A-B shows miscibility of IND and Polymer based on Flory-Huggins theory: Variation of the interaction parameter, χ, as a function of temperature (FIG. 10A); Plot of ΔGₘᵢₓ/RT as a function of drug volume fraction, ϕ, for IND and polymers at 150° C. (FIG. 10B).

FIG. 11A-H shows SEM data of HM processed particles containing XDP. XDP: 500× (FIG. 11A), 10,000× (FIG. 11B); IND:AF15:XDP formulation: 500× (FIG. 11C), 10,000× (FIG. 11D); IND:VA64:XDP formulation: 500× (FIG. 11E), 10,000× (FIG. 11F); and IND:KIR:XDP formulation: 500× (FIG. 11G), 10,000× (FIG. 11H).

FIGS. 12A-D show powder X-ray diffraction (FIG. 12A) and mDSC (FIG. 12C) for formulations 8 and 10 (shown in Table 10) as well as powder X-ray diffraction (FIG. 12B) and mDSC (FIG. 12D) for formulations 9 and 11 (shown in Table 5). Results shown are for nifedipine as the therapeutic agent.

FIGS. 13A-B shows dissolution profiles (n=3) of formulations 8 and 10 (FIG. 13A) as well as of formulations 9 and 11 (FIG. 13B). Formulation 8-11 are shown in Table 5; and the results shown are for nifedipine as the therapeutic agent.

FIGS. 14A-B shows powder X-ray diffraction (FIG. 14A) and mDSC (FIG. 14B) for formulations 12 and 13 (shown in Table 11). Results shown are for ritonavir as the therapeutic agent.

FIG. 15 shows dissolution profiles (n=3) of formulations 12 and 13. Formulation 12 and 13 are shown in Table 11; and the results shown are for ritonavir as the therapeutic agent.

FIGS. 16A-B shows powder X-ray diffraction (FIG. 16A) and mDSC (FIG. 16B) for formulations 14 and 15 (shown in Table 12). Results shown are for itraconazole as the therapeutic agent.

FIG. 17 shows dissolution profiles (n=3) of formulations 14 and 15. Formulation 14 and 15 are shown in Table 12; and the results shown are for itraconazole as the therapeutic agent.

FIG. 18 shows appearance of formulations 16 and 17 after HME process. Formulation 16 and 17 are shown in Table 13; and the results shown are for itraconazole as the therapeutic agent.

FIG. 19 shows dissolution profiles (n=2) of formulations 16 and 18. Formulation 16 and 18 are shown in Table 13; and the results shown are for itraconazole as the therapeutic agent.

FIG. 20 shows mDSC (FIG. 20) for formulations 18 before and after 6 months storage in a desiccator at room temperature (shown in Table 12). Results shown are for itraconazole as the therapeutic agent.

FIG. 21 shows dissolution profiles (n=2) of formulations 18 before and after 6 months storage in a desiccator at room temperature. Formulation 18 is shown in Table 13; and the results shown are for itraconazole as the therapeutic agent.

### DESCRIPTION OF ILLUSTRATIVE EMBODIMENTS

In some aspects of the present disclosure, the pharmaceutical compositions prepared through thermal processing and containing a therapeutic agent, a pharmaceutically acceptable polymer, and a mesoporous carrier which show improved solubility or other pharmaceutical properties are provided. These compositions may show improved solubility parameters and exhibit “spring and parachute” dissolution relative to other compositions. In some embodiments, the compounds exhibit an increased initial concentration with only a slow taper in the overall solution concentration over the course of hours. In some embodiments, these compositions comprise a mesoporous carrier wherein the therapeutic agents are dissolved, absorbed, or present on both the inside of the pores of the carrier as well as the carrier surface. Additionally, the choice of polymer may be guided by using such parameters as Flory-Huggins theory to predict the specific pairing of the therapeutic agent and the pharmaceutically acceptable polymer. In particular, drug-polymer systems containing high positive χ values may demonstrate improved properties relative to compositions despite such high χ value systems to be considered metastable to unstable and typically avoided. Also provided herein are methods of preparing and using these compositions. Details of these compositions are provided in more detail below.

### I. PHARMACEUTICAL COMPOSITIONS

In some aspects, the present disclosure provides pharmaceutical compositions containing a therapeutic agent, a pharmaceutically acceptable polymer, and a mesoporous carrier which have been processed through a thermal process or fusion-based high energy mixing process. In some embodiments, the thermal process may be a hot melt extrusion or a hot melt granulation process. In other embodiments, the fusion-based high energy process is a process which results in an increase in temperature without requiring an external heat input including thermokinetic mixing process such as those described in U.S. Pat. Nos. 8,486,423; 9,339,441; Prasad et al., 2016; LaFountaine et al., 2016; and DiNunzio et al., 2010d. Additionally, these pharmaceutical compositions may show improved solubility or dissolution profiles which result in one or more improved therapeutic parameters or outcomes.

These pharmaceutical compositions may be used and prepared in the absence of a solvent. As used herein a solvent is used within its conventional meaning as a liquid phase component that dissolves one or more components such that those compounds are partially or fully dissolved to form a homogenous mixtures. In one embodiments, the pharmaceutical compositions are prepared in the absence of an organic solvent which may be used to pre-load the mesoporous carrier. The present pharmaceutical composition may have the advantage that the formulation does not require the use of loading of the mesoporous carrier through another step such as with a solvent.

Additionally, the present pharmaceutical composition may have the added benefit of not requiring the mixing or milling of the components of the composition before being subjected to the thermal or fusion-based high energy processes. Such advantages simplify the formulation process and reduce the possible likelihood of drug decomposition or degradation. In some embodiments, the present compositions may also have the advantage that they allow the processing of the components at a lower temperature to obtain or maintain a lack of crystallinity relative to compositions which contain either the pharmaceutically acceptable polymer or the mesoporous carrier.

A. Therapeutic Agent

The “therapeutic agent” used in the present methods and compositions refers to any substance, compound, drug, medicament, or other primary active ingredient that provides a therapeutic or pharmacological effect when administered to a human or animal. Some non-limiting examples of lipophilic therapeutic agents are BCS classes II and IV compounds or other agents that similarly exhibit poor solubility. The BCS definition describes a compound in which the effective dosing is not soluble in 250 mL of water at a pH from 1-7.5. The USP categories “very slightly soluble” and “insoluble” describe a material that requires 1,000 or more parts of the aqueous liquid to dissolve 1 part solute. As used herein, when a compound is described as poorly soluble, it refers to a compound that has solubility in water of less than 1 mg/mL. In other embodiments, the therapeutic agent is an active agent that has a high melting point. Some non-limiting examples of high melting point therapeutic agents are griseofulvin and theophylline.

When a therapeutic agent is present in the composition, the therapeutic agent is present in the composition at a level between about 50% to 98% w/w, between about 50% to 90% w/w, between about 50% to 80% w/w, between about 50% to 75% w/w, or between about 50% to 60% w/w of the total composition. In some embodiments, the amount of the therapeutic agent is from about 50%, 51%, 52%, 53%, 54%, 55%, 56%, 57%, 58%, 59%, 60%, 62%, 64%, 65%, 66%, 68%, 70%, 72%, 74%, 75%, 76%, 78%, 80%, 85%, 90%, 95%, to about 98% w/w or any range derivable therein.

Suitable therapeutic agents, including lipophilic therapeutic agents may be any poorly water-soluble, biologically active agents or a salt, isomer, ester, ether or other derivative thereof, which include, but are not limited to, anticancer agents, antifungal agents, psychiatric agents such as analgesics, consciousness level-altering agents such as anesthetic agents or hypnotics, nonsteroidal anti-inflammatory drugs (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the therapeutic agents may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albendazole, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine HCl, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin (neutral), doxorubicin HCl, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, mebendazole, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, omidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the therapeutic agents may be busulfan, taxane or other anticancer agents; or alternatively, itraconazole (Itra) and posaconazole (Posa) or other members of the general class of azole compounds. Exemplary antifungal azoles include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, Posaconazole, voriconazole, terconazole and c) thiazoles such as abafungin. Other drugs that may be used with this approach include, but are not limited to, hyperthyroid drugs such as carimazole, anticancer agents like cytotoxic agents such as epipodophyllotoxin derivatives, taxanes, bleomycin, anthracyclines, as well as platinum compounds and camptothecin analogs. The following therapeutic agents may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The agents may also include a psychiatric agent such as an antipsychotic, anti-depressive agent, or analgesic and/or tranquilizing agents such as benzodiazepines. The agents may also include a consciousness level-altering agent or an anesthetic agent, such as propofol. The present compositions and the methods of making them may be used to prepare a pharmaceutical compositions with the appropriate pharmacokinetic properties for use as therapeutics.

In some aspects, the method may be most used with materials that undergo degradation at an elevated temperature or pressure. The therapeutic agents that may be used include those which decompose at a temperature above about 50° C. In some embodiments, the therapeutic agent decomposes above a temperature of 80° C. In some embodiments, the therapeutic agent decomposes above a temperature of 100° C. In some embodiments, the therapeutic agent decomposes above a temperature of 150° C. The therapeutic agent that may be used include therein which decompose at a temperature of greater than about 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., or 150° C.

B. Pharmaceutically Acceptable Polymers

In some aspects, the present disclosure provides compositions which may further comprise a pharmaceutically acceptable polymer. In some embodiments, the polymer has been approved for use in a pharmaceutical formulation and is known to undergo softening or increased pliability when raised above a specific temperature without substantially degrading. Additionally, the pharmaceutically acceptable polymer may also be known to enhance the dissolution of one or more of the therapeutic agents in the composition or pharmaceutical composition.

When a pharmaceutically acceptable polymer is present in the composition, the pharmaceutically acceptable polymer is present in the composition at a level between about 1% to about 49% w/w, between about 5% to about 45% w/w, between about 10% to about 40% w/w, between about 20% to about 40% w/w, between about 20% to about 30% w/w of the total pharmaceutical composition or the total composition. In some embodiments, the amount of the pharmaceutically acceptable polymer is from about 1%, 5%, 10%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 45%, 48%, to about 49% w/w or any range derivable therein.

In some aspects, Flory-Huggins theory can be used as a preformulation test to guide or predict appropriate therapeutic agent and pharmaceutically acceptable polymer combination. Flory-Huggins theory may be used to predict miscibility information for amorphous drug-polymer systems by evaluating the drug-polymer interaction parameter, χ, to calculate the free energy of mixing (ΔGₘᵢₓ) for the system. The χ value stems from the non-ideal entropy of mixing of the pharmaceutically acceptable polymer molecule with the therapeutic agent and takes into account the contribution due to the enthalpy of mixing (Bansal et al., 2016). More negative χ values predict miscibility whereas more positive χ values predict immiscibility of the therapeutic agent-polymer system (Bansal et al., 2016; Marsac et al., 2006). According to Flory-Huggins theory,

$$Δ {G}_{mix} = RT ( {Φ}_{drug} ln {Φ}_{drug} + \frac{{Φ}_{polymer}}{m} ln {Φ}_{polymer} + {χΦ}_{drug} {Φ}_{polymer} ) \qquad ( Equation 1 )$$

where Φ is the volume fraction, χ is the Flory-Huggins interaction parameter, R is the molar gas constant, and T is the temperature. m is the ratio of the volume of a pharmaceutically acceptable polymer to the therapeutic agent molecular volume and,

$$m = \frac{{MW}_{polymer} \text{/} {ρ}_{polymer}}{{MW}_{drug} \text{/} {ρ}_{drug}} \qquad ( Equation 2 )$$

where the MW_{polymer} and MW_{drug} are the molecular weight of the pharmaceutically acceptable polymer and therapeutic agent, respectively, and the ρ_{polymer} and ρ_{drug} are the density of pharmaceutically acceptable polymer and therapeutic agent, respectively. The primary method for determining the χ value is by analyzing the melting point depression of the solid dispersion system, which is often, analyzed using differential scanning calorimetry (DSC). DSC is utilized to determine the melting point onset (Zhao et al., 2011), melting temperature (Lin and Huang, 2010; Marsac et al., 2008), or melt endpoint (Tian et al., 2013). Following analysis of melting point depressions, the χ value can be calculated using the following rearranged equation (Marsac et al., 2006).

$$( \frac{1}{{T}_{M}^{mix}} - \frac{1}{{T}_{M}^{pure}} ) ( \frac{Δ {H}_{fus}}{- R} ) - ln {Φ}_{drug} - ( 1 - \frac{1}{m} ) {Φ}_{polymer} = {χΦ}_{polymer}^{2} \qquad ( Equation 3 )$$

where T_{M} values are the melting points of the mixture of pure therapeutic agent, R is the ideal gas constant, ΔH_{fus} is the heat of fusion for the pure therapeutic agent, m is a constant for the relative size of the pharmaceutically acceptable polymer to the therapeutic agent, and the Φ values are volume fraction of therapeutic agent or pharmaceutically acceptable polymer. If the plot of the left side of the rearranged equation vs. the Φ² value for the pharmaceutically acceptable polymer demonstrates linearity, the slope of the best-fit line is considered to be equivalent to χ. By understanding χ as a function of temperature, metastable and unstable regions for the combination can be predicted by generating a spinodal (boundary between unstable and metastable regions) and binodal (boundary between metastable and stable regions) curves (Huang et al., 2016). If the solid dispersion system's components are stable, these systems tend to remain in a single-phase, while metastable and unstable systems tend to phase separate into drug-rich and polymer-rich domains upon storage. Without wishing to be bound by any theory it is believed that the tendency to recrystallize occurs because the high-energy amorphous state is generally unstable (Marsac et al., 2010; Purohit and Taylor, 2015). Therefore, in some embodiments, Flory-Huggins theory as a preformulation test contemplates that the combination of the pharmaceutically acceptable polymer and the therapeutic agent exhibits a stable combination. In other aspects, the present combinations of the pharmaceutically acceptable polymer and the therapeutic agent exhibits a positive χ value.

Within the compositions described herein, a single polymer or a combination of multiple polymers may be used. In some embodiments, the polymers used herein may fall within two classes: cellulosic and non-cellulosic. These classes may be further defined by their respective charge into neutral and ionizable. Ionizable polymers have been functionalized with one or more groups, which are charged at a physiologically relevant pH. Some non-limiting examples of neutral non-cellulosic polymers include polyvinyl pyrrolidone, polyvinyl alcohol, copovidone, poloxamer, polyethylene oxide, polypropylene oxide, polyvinylpyrrolidone-co-vinylacetate, polyethylene, polycaprolactone, and polyethylene-co-polypropylene. Some examples of ionizable non-celluolosic polymers include polymethacrylate or polyacrylate such as Eudragit®. Some non-limiting examples of ionizable cellulosic polymers include hydroxyalkylalkyl cellulose ester such as cellulose acetate phthalate and hydroxypropyl methyl cellulose acetate succinate, carboxyalkyl cellulose such as carboxymethyl cellulose and alkali metal salts thereof, such as sodium salts, and carboxyalkylalkyl cellulose including carboxymethylethyl cellulose, carboxyalkyl cellulose ester such as carboxymethyl cellulose butyrate, carboxymethyl cellulose propionate, carboxymethyl cellulose acetate butyrate, and carboxymethyl cellulose acetate propionate. Finally, some non-limiting examples of neutral cellulosic polymers include alkylcelluloses such as methylcellulose, hydroxyalkylcelluloses such as hydroxymethylcellulose, hydroxypropyl cellulose, hydroxyethylcellulose, and hydroxybutylcellulose, hydroxyalkyl alkylcelluloses such as hydroxyethyl methylcellulose and hydroxypropyl methyl cellulose, starches, pectins, chitosan or chitin and copolymers and mixtures thereof, and polysaccharides such as tragacanth, gum arabic, guar gum, and xanthan gum.

Some specific pharmaceutically acceptable polymers which may be used include, for example, Eudragit™ RS PO, Eudragit™ S100, Kollidon SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), carboxymethyl cellulose and alkali metal salts thereof, such as sodium salts sodium carboxymethyl-cellulose (CMC), dimethylaminoethyl methacrylate-methacrylic acid ester copolymer, carboxymethylethyl cellulose, carboxymethyl cellulose butyrate, carboxymethyl cellulose propionate, carboxymethyl cellulose acetate butyrate, carboxymethyl cellulose acetate propionateethylacrylate-methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, poly(methacylic acid-co-methyl methacrylate 1:2), poly(methacrylic acid-co-methyl methacrylate 1:1), Poly(methyl acrylate-co-methyl methacrylate-co-methacrylic acid 7:3:1), poly(butyl methacrylate-co-(2-dimethylaminoethyl) methacrylate-co-methyl methacrylate 1:2:1), poly(ethyl acrylate-co-methyl methacrylate 2:1), poly(ethyl acrylate-co-methyl methacrylate 2:1), poly(ethyl acrylate-co-methyl methacrylate-co-trimethylammonioethyl methacrylate chloride 1:2:0.2), poly(ethyl acrylate-co-methyl methacrylate-co-trimethylammonioethyl methacrylate chloride 1:2:0.1), Eudragit L-30-D™ (MA-EA, 1:1), Eudragit L-100-55™ (MA-EA, 1:1), hydroxypropylmethylcellulose acetate succinate (HPMCAS), polyvinyl caprolactam-polyvinyl acetate-PEG graft copolymer, polyvinyl alcohol/acrylic acid/methyl methacrylate copolymer, polyalkylene oxide, Coateric™ (PVAP), Aquateric™ (CAP), and AQUACOAT™ (HPMCAS), polycaprolactone, starches, pectins, chitosan or chitin and copolymers and mixtures thereof, and polysaccharides such as tragacanth, gum arabic, guar gum, and xanthan gum.

C. Mesoporous Carrier

In some aspects, the present disclosure contemplates the use of one or more mesoporous carriers. A mesoporous carrier is a porous material containing pore diameters from about 2 to about 50 nm. In some embodiments, the mesoporous carrier may be prepared using polymeric or inorganic materials. The mesoporous carriers used herein may be those prepared using inorganic materials such as silica, alumina, carbon, zirconia, metal oxides, or mixtures thereof. In one embodiment, mesoporous materials of silica are used in the compositions herein including both order and non-ordered silica or mixtures thereof. Examples of mesoporous carrier, their characteristics, and their preparation are described in Sayed et al., 2017 and Maleki et al., 2017, both of which are incorporated herein by reference.

Some of the mesoporous materials used herein may have a diameter of less than 1 μm, including from about 10 nm to about 500 nm or from about 50 nm to about 250 nm. Additionally, the pore size of these materials may be from about 2 nm to about 100 nm, from about 5 nm to about 50 nm, or from about 5 nm to about 25 nm. Additionally, it is contemplated that the mesoporous carriers may be functionalized with one or more polymers or lipids to modify the properties of the mesoporous carriers. Additionally, the mesoporous carriers that may be used in this study have not been preloaded with the therapeutic agent before formulation with the pharmaceutically acceptable polymer. In some embodiments, the mesoporous carrier has not been preloaded with a therapeutic agent by solvent evaporation, incipient wetness, or melt before the mesoporous carrier is processed with the therapeutic agent and the pharmaceutically acceptable polymer.

Some non-limiting examples of mesoporous carriers which may be used in the present pharmaceutical composition include silica (SiO₂), e.g. Syloid® like Syloid® AL-1FP or Syloid® 72FP, alumina, magnesium alumino-metasilicates like Al₂O₃.MgO.1.7SiO₂.xH₂O, (Neusilin® US2) or other mixed inorganic oxides, CaCO₃, clay, or other materials including those in WO 2012/072580 and WO 2014/078435, which are both incorporated herein by reference, such as SBA-15 mesoporous silica, SBA-16, MCM-41, COK-12. KIT-6, or FDU-12.

In some aspects, the amount of mesoporous carrier is from about 1% to about 49% w/w. The amount of mesoporous carrier comprises from about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, to about 49% w/w, or any range derivable therein, of the total pharmaceutical composition. In one embodiment, the amount of mesoporous carrier is at 20 to 30% w/w of the total weight of the pharmaceutical composition.

### II. THERMAL METHODS

Thus, in one aspect, the present disclosure provides pharmaceutical compositions which may be prepared using a thermal or fusion-based high energy process. Such process may include hot melt extrusion, hot melt granulation, melt mixing, spray congealing, sintering/curing, injection molding, or a thermokinetic mixing process such as the KinetiSol method. Similar thermal processing methods are described in LaFountaine et al., 2016a, Keen et al., 2013, Vynckier et al., 2014, Lang et al., 2014, Repka et al., 2007, Crowley et al., 2007, DiNunzio et al., 2010a, DiNunzio et al., 2010b, DiNunzio et al., 2010c, DiNunzio et al., 2010d, Hughey et al., 2010, Hughey et al., 2011, LaFountaine et al., 2016b, and Prasad et al., 2016, all of which are incorporated herein by reference. In some embodiments of these present disclosure, the pharmaceutical compositions may be prepared using a thermal process such as hot melt extrusion or hot melt granulation. In other embodiments, a fusion based process including thermokinetic mixing process such as those described at least in U.S. Pat. Nos. 8,486,423 and 9,339,440, the entire contents of which are herein incorporated by reference.

A non-limiting list of instruments which may be used to thermally process the pharmaceutical compositions described herein include hot melt extruders available from ThermoFisher, such as a minilab compounder, or Leistritz, such as a twin-screw extruder. Alternatively, a fusion-based high energy process instrument that does not require external heat input, including such as a thermokinetic mixer as described in U.S. Pat. Nos. 8,486,423 and 9,339,440 may be used to process the pharmaceutical composition.

In some aspects, the extruder may comprise heating the composition to a temperature from about 60° C. to about 250° C. In some embodiments, the temperature is from about 100° C. to about 200° C. The temperature that may be used is from about 60° C., 65° C., 70° C., 75° C., 80° C., 90° C., 92° C., 94° C., 96° C., 98° C., 100° C., 102° C., 104° C., 106° C., 108° C., 110° C., 112° C., 114° C., 116° C., 118° C., 120° C., 125° C., 130° C., 135° C., 140° C., 145° C., 150° C., 155° C., 160° C., 165° C., 170° C., 175° C., 180° C., 190° C., 200° C., 225° C., to about 250° C. or any range derivable therein.

The extrudate produced following the extrusion process will generally comprise the therapeutic agent, the mesoporous carrier and the pharmaceutically acceptable polymer. The extrudate may be in the form of granules of a desired mesh size or diameter, rods that can be cut and shaped into tablets, and films of a suitable thickness that shaped forms can be punched into suitable size and shape for administration. This extrudate may be used in further processing steps to yield the final pharmaceutical product or composition. The extrudate of the pharmaceutical composition may be dried, formed, milled, sieved, or any combination of these processes to obtain a final composition which may be administered to a patient. Such processes are routine and known in the art and include formulating the specific product to obtain a final pharmaceutical or nutraceutical product. Additionally, the extrudate of the pharmaceutical composition obtained may be processed using a tablet press to obtain a final table. Additionally, it may be milled and combined with one or more additional excipients to form a capsule or pressed into a tablet. The resultant pharmaceutical composition may also be dissolved in a pharmaceutically acceptable solvent to obtain a syrup, a suspension, an emulsion, or a solution.

### III. EXAMPLES

To facilitate a better understanding of the present invention, the following examples of specific embodiments are given. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. In no way should the following examples be read to limit or define the entire scope of the invention.

### Example 1—Materials and Methods

Preformulation and formulation studies were conducted as follows:

Materials.

Hydrous silicon dioxide (Syloid® XDP 3050, XDP) was donated by Grace Japan K.K. (Japan). Hypromellose (Affinisol™ HPMC HME 15LV, AF15) was donated by The Dow Chemical Company (Midland, Mich., USA). Copovidone (Kollidon® PVP VA64, VA64) and polyvinyl alcohol—polyethylene glycol graft copolymer (Kollicoat® IR, KIR) were donated by BASF The Chemical Company (Florham Park, N.J., USA). Indomethacin, USP (IND) was purchased from HAWKINS (Minneapolis, Minn., USA). Other chemicals were of reagent grade.

### Methods.

Sample Preparation:

Exemplary thermal/shear processing techniques were employed. Among these thermal processes, a hot melt extrusion (HME) process was employed, which required heat and high shear force, and a hot melt (HM) process (a type of hot melt granulation process), which required heat and low shear force. Formulations 1-3 and 7 (Table 1) were prepared using the aforementioned HM technique. HM was as follows: the formulations were mixed until uniform using a mortar and pestle then heated at 150° C. for 10 minutes in a Breville Smart Oven® Pro (Breville USA, Torrance Calif.). The cooled sample was prepared by using a mortar and pestle to a uniform granule size. Formulation 6 (Table 1) was processed utilizing the same conditions but then milled with a grinder to size the granulated/aggregated material obtained during the hot melt technique. The formulation was sieved through a 212 μm screen and collected. Formulations 4 and 5 (Table 1) were processed by HME using a co-rotating HAAKE Minilab II (Thermo Electron Corporation, Newington, N.H.). The formulations were extruded at 150° C. and a screw speed of 100 rpm. Extrudates were cooled to room temperature before further processing. The cooled extrudates were milled with a grinder and sieved through a 212 μm screen and collected. In addition, formulation 1 was processed by HME using a Leistritz Nano-16 co-rotating, twin-screw extruder (American Leistritz Extruder Corp., Somerville, N.J.) equipped with twin-screws containing kneading elements (30° and 60°) and without a die. Conveying, kneading, and mixing elements were used in the screw design, and each operation conditions are illustrated in Table 2. A twin-screw volumetric feeder (Brabender Technology, Duisburg, Germany) set on top of the barrel feed zone provided an accurate 1 g/min feed rate of the powder blend that was mixed until uniform. The barrel configuration consisted of a feed zone, closed barrel, closed venting zone, and a closed zone before the top block. The feeding zone was maintained at room temperature conditions with water circulation. The barrel temperatures for zones 1, 2, and 3 were 150° C., 150° C., and 150° C., respectively. All extrudates were cooled to room temperature, and then milled and sieved through a 212 μm screen (see, Hanada et al, 2018 (2)).

TABLE 1
Binary and ternary formulation ratios used for thermal processing

| | | | | | | | Ternary |
|---|---|---|---|---|---|---|---|
| | | | | | | | Formulation |
| | | | | | | | (IND:Polymer: |
| Formu- | Thermal | IND | AF15 | VA64 | MR | XDP | XDP) |
| lation | Process | (0/0) | (0/0) | (0/0) | (0/0) | (0/0) | (by weight) |
| 1 | HM | 50 | 20 | — | — | 30 | 5:2:3 |
| 2 | HM | 50 | — | 20 | — | 30 | 5:2:3 |
| 3 | HM | 50 | — | — | 20 | 30 | 5:2:3 |
| 4 | HME | 71.4 | 28.6 | — | — | — | 5:2:0 |
| 5 | HME | 71.4 | — | 28.6 | — | — | 5:2:0 |
| 6 | HM | 71.4 | — | — | 28.6 | — | 5:2:0 |
| 7 | HM | 50 | — | — | — | 50 | 5:0:5 |

Modulated Differential Scanning Calorimetry (mDSC):

To characterize the thermal behavior of the samples, mDSC equipped with a DSC refrigerated cooling system (DSC 2920, TA Instruments, New Castle, Del.) was employed. Dry nitrogen gas at a flow rate of 40 mL/min throughout the testing was used to purge the DSC cell. Samples were accurately weighed in aluminum sample pan kits (PerkinElmer Inc., Shelton, Conn.) and crimped before analysis. Samples were heated from 25° C. to 200° C. with a heating ramp rate of 10° C./min using a 1° C./60 sec. modulation program. TA Universal Analysis 2000 software was used to process the raw data.

Thermogravimetric Analysis (TGA):

TGA was performed on a TGA/DSC 1 (Mettler Toledo, Schwerzenbach, Switzerland). The temperature ramp utilized in this experiment was performed from 25° C. to 300° C. at a rate of 5° C./min with a nitrogen purge at 50 mL/min. Data was analyzed using STARe System.

Powder X-Ray Diffraction (PXRD):

PXRD studies were conducted on a Rigaku Miniflex600 II (Rigaku Americas, The Woodlands, Tex.) instrument equipped with a Cu-Kα radiation source generated at 40 kV and 15 mA. The 2-theta angle, step size, and scan speed were set to 5°-40°, 0.02°, and 5°/min, respectively. 2°/min was used for crystallinity calculation. In order to obtain PXRD patterns, the raw data was processed using PDXL2 software (Rigaku Americas, The Woodlands, Tex.).

Physical Stability: About 1 g of each ternary ASD was accurately weighed into a glass vial and stored in a sealed desiccator at 40° C./75% RH (saturated sodium chloride solution) without protection from moisture exposure. Samples were analyzed by PXRD and mDSC at time points of 0, 1, 3, 7 and 14 days as described above. After analysis, only PXRD samples were immediately returned to the desiccator for storage. In PXRD, the relative crystallinity was calculated by dividing all crystalline peaks area in the sample by all crystalline peaks area in physical mixture (PM). Also, using mDSC, the relative crystallinity was calculated by dividing heat of fusion (ΔH) of the endothermic event in the extruded sample by ΔH of endothermic event of the PM. The relative crystallinity of PM was defined as 100% (see, Hanada et al, 2018 (2)).

Scanning Electron Microscopy (SEM):

Samples were mounted on standard aluminum SEM stubs and sputter coated with 12 nm platinum/palladium (Pt/Pd) using a Cressington sputter coater 208HR (Cressington Scientific Instruments Ltd., Watford, UK) and were imaged using a Zeiss Supra 40VP SEM (Carl Zeiss Microscopy GmbH, Jena, Germany).

Specific Surface Area (SSA):

The SSA was determined with a single-point BET method using a Monosorb® surface area analyzer (Quantachrome, Boynton Beach, Fla.). Approximately 100 mg of pure XDP was carefully weighed and added to a tared glass sample holder and allowed to degas for 24 hours at 105° C. The formulations containing XDP weighed approximately 100 mg, and the formulations without XDP weighed approximately 200 mg and were added to the tared glass sample holder. The IND-loaded samples were allowed to degas for 20 hours at 40° C. BET nitrogen adsorption and desorption was performed using a 30% v/v mixture of nitrogen in helium. SSA values were determined from desorption of nitrogen.

Rheology:

Rheology experiments were performed with a TA AR-2000ex Rheometer using an attached Environmental Test Chamber (ETC) (New Castle, Del.). Samples were prepared as previously described (Gupta et al., 2014) by weighing out approximately 1 g of material and pressing into a slug using a 25 mm die geometry and hydraulic press with 5000 psi of force for 5 seconds. The sample was placed between two parallel 25 mm steel plates after zero gap calibration. The ETC was equilibrated at 150° C. before inserting the drug-polymer slug between the plates. A time sweep was performed for 10 min at 150° C. and angular velocity 0.1 rad/s. A strain of 0.5% was used along with an axial force control of 10 N±5 N to ensure adequate contact with the plates. Rheology was performed for IND:AF15, IND:VA64, and IND:KIR samples in a 5:2 ratio of IND:polymer.

Specific Mechanical Energy (SME):

Among the parameters in a twin-screw extruder, screw speed and feed rate affect the SME input (Haser et al., 2017). Furthermore, it is understood that the screws generate the majority of the energy used to dissolve drug in polymer, even though the extruder barrels are heated (Haser et al., 2017; Brown et al., 2014). Generally, SME demonstrates the amount of power that is contributed by the twin-screw extruder motor per kilogram of material being processed. SME was calculated by a two-step equation, as shown below (Martin, 2008; Huang et al., 2017; Hanada et al, 2018 (2)):

### Applied Power

$$KW ( applied ) = KW ( motor rating ) \times \% Torque \times \frac{{rpm}_{running}}{{rpm}_{max}} \times 0.97 ( gearbox efficiency ) \qquad ( Equation 4 )$$

### Specific Mechanical Energy (SME)

$$SME = \frac{{KW}_{( applied )}}{Feed Rate} \qquad ( Equation 5 )$$

Dissolution:

Dissolution testing was performed at non-sink conditions. A Hanson SR8PLUS dissolution apparatus (Hanson Research Co., Chatsworth, Calif.) with corresponding paddles was utilized to perform the testing according to USP Apparatus II. The paddle speed and temperature were set to 100 rpm and 37° C.±0.5° C., respectively. Before testing, 900 mL of deionized water was pre-heated to 37° C. in each dissolution vessel. 212 μm sieve-passed samples containing 200 mg IND equivalent (n=3) were then added immediately to the dissolution vessel. A 2 mL sample was collected at time points 15, 30 min, 1, 2, 4, and 6 hours for HM and HME samples prepared by an oven and co-rotating HAAKE Minilab II, respectively for 15, 30 min, 1, 2, 4, 6, 8, 12, 16, 20 and 24 hours for SME samples prepared by Nano-16 with twin-screw. The sample was pulled and filtered through a 0.45 μm 25 mm PES membrane filter. A 500 μL aliquot of filtered solution was diluted with 500 μL HPLC grade acetonitrile, and the concentration of IND in the diluted sample was measured using HPLC.

High Performance Liquid Chromatography (HPLC) (Nováková et al., 2005):

IND was detected by a Breeze HPLC system (Waters, Milford, Mass.) equipped with a ZORBAX® CN (5 μm, 4.6×150 mm) column. The composition of HPLC mobile phase was 50/50/1 acetonitrile/water/phosphoric acid. The mobile phase flow rate was 1 mL/min and detection wavelength 237 nm. In quantitative analysis, the milled and sieved samples were accurately weighed to 40 mg and transferred to volumetric flasks in triplicate to prepare 100 μ/mL solutions of IND. A 50:50 volume ratio of HPLC grade acetonitrile to deionized water mixture was used as the diluent. Diluent was added to the volumetric flask and sonicated before filling to volume. The IND solutions were left to stand and 500 μL of the supernatant was diluted with 500 μL of diluent and then transferred to HPLC vials for analysis.

Particle Size Distribution:

Particle size distribution was conducted in accordance with the method reported by Ellenberger et al. (2018). The particle size distribution (PSD) of the milled and sieved samples was analyzed using a Spraytec analyzer (Malvern Instruments, Malvern, UK). Each sample was pre-loaded into a size 3 gelatin capsule and the capsule was subsequently punctured to allow for sample exit and air flow escape. A feed pressure of 60 psi dry nitrogen was used to administer the powder into the unit.

Flory-Huggins Modeling:

IND and polymer were prepared at different ratios with a total weight of 100 mg. The samples were suspended in 1.5 mL anhydrous ethanol and stirred using a vortex mixer. The suspension was transferred to an evaporating dish and washed with 0.75 mL ethanol. The sample suspension was evaporated using a drying oven overnight. For the thermal analysis, polymer effects were evaluated at 0, 10, 15, 20, 25, 30, 35, and 40% w/w polymer in the drug-polymer mixture. Each sample was accurately weighed in aluminum sample pan kits (PerkinElmer Inc., Shelton, Conn.) and crimped before analysis. DSC was performed using the DSC 2920 instrument mentioned previously (TA Instruments, New Castle, Del.). The end melting temperatures of IND were observed as samples were heated from 50° C. to 180° C. with a heating rate of 10° C./min. Dry nitrogen gas at a flow rate of 40 mL/min throughout the testing was used to purge the DSC cell. TA Universal Analysis 2000 software was used to process the raw data.

Solid-State NMR (ssNMR):

ssNMR experiments were performed using a 500 MHz Bruker Avance III spectrometer in the Pharmaceutical NMR lab of Preclinical Development at Merck Research Laboratories (Merck & Co., Inc. West Point, Pa.). Without being bound to a specific theory, ssNMR was used to support the characterization of the benefits that the compositions provide. One-dimensional (1D) and two-dimensional (2D) spectra for ¹H, ¹³C and ²⁹Si were obtained at magic angle spinning (MAS) of 12 kHz with a Bruker 4 mm HFX MAS probe in double-resonance mode tuned to ¹H and X-nucleus frequencies (where the X-nucleus was either ¹³C or ²⁹Si). ¹H, ¹³C and ²⁹Si spectra were referenced to the tetramethylsilane (TMS) using as an external reference sample. All spectra were acquired at 298 K and processed in Bruker Topspin 3.5 software. The 90-degree pulse duration was set to 3 μs for ¹H excitation. 1D ¹³C cross-polarization (CP) transfers were performed with radio-frequency (RF) strength of 80-100 kHz during a 2 ms contact time. The power level was ramped linearly over a depth of 15 to 20 kHz on the ¹H channel to enhance CP efficiency. 1D ²⁹Si CP transfers were performed with a 5 ms contact time. ¹H heteronuclear decoupling for ¹³C and ²⁹Si were performed at RF strength of 100 kHz using the SPINAL-64 pulse sequence. 2D heteronuclear dipolar correlation (HETCOR) experiments between ¹H and ¹³C, as well as ¹H and ²⁹Si nuclei were obtained with contact time of 2 and 5 ms, respectively, to obtain long-range intermolecular correlation peaks revealing structure and interaction information. Relaxation measurements (Yuan et al., 2014; Yang et al., 2016; Purohit et al., 2017) were performed at MAS of 12 kHz. Briefly, ¹H spin-lattice relaxation times in the laboratory frame (T₁) were determined by ¹³C-detected saturation recovery experiments with fitting of 10 time points using the following exponential function:

I(τ)=I₀×(1−e^{-τ/T1}) (Equation 6)

Where τ is the recovery delay time point, I(τ) is the peak intensity of each resolved peak at the time point τ, I₀ is a scaling factor of signal intensity from the fit, and T₁ is the spin-lattice relaxation time to be derived. ¹H spin-lattice relaxation times in the rotating frame (T_{1ρ}) were determined by applying a variable spin-lock pulse at RF strength of 100 kHz following the ¹³C CP experiments. The T_{1ρ} were derived with fitting of 11 time points using the following exponential function:

I(τ)=I₀×e^{-τ/T1τ} (Equation 7)

Where τ is the spin-lock duration, I₀) is the peak intensity of each resolved peak at the time point τ, and I₀ is a scaling factor of signal intensity from the fit (see, Hanada et al, 2018 (2)).

### Example 2—Processing Conditions

Based on Example 1, the processing conditions and results are as follows:

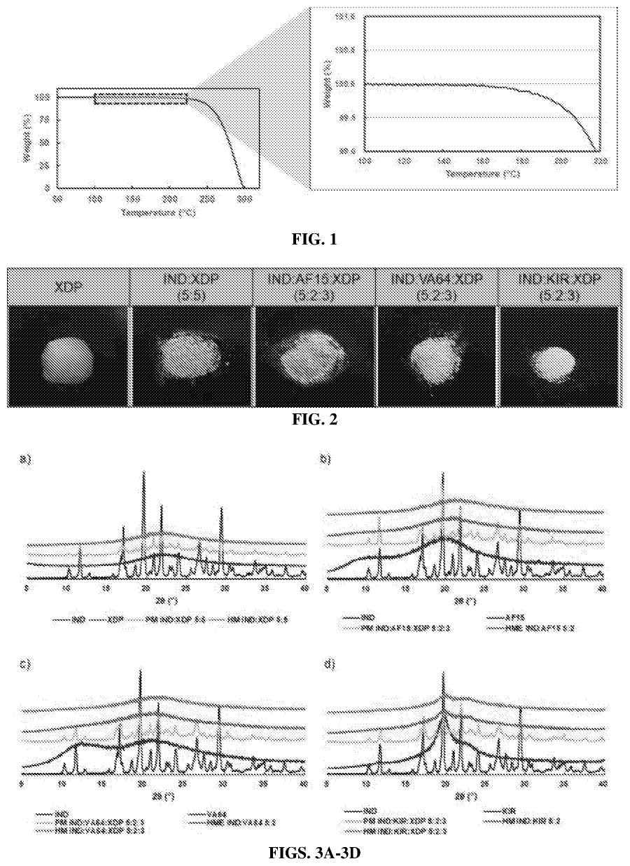

Thermogravimetric analysis was used to investigate the onset of thermal degradation for IND and assisted with determination of the upper limit of temperature when processing. IND exhibited weight loss as a result of degradation with an onset near the drug's melting point of 160° C. (FIG. 1). Based on the TGA results, the upper limit for processing temperature was 160° C. For processing, it is also important to take into consideration the melting point of IND. Without wishing to be bound by any theory, in order to cover the IND-polymer mixture onto particles XDP by our thermal processing methods, the temperature conditions implemented should be carried out below the melting point of IND. However, it is possible to lower the melting point of IND, since it was observed that mixing IND and polymer resulted in melting point depression. Therefore, the processing temperature was set at 150° C., which is both below the melting point of IND and the temperature where degradation was observed.

Samples of (5:2:3) IND:AF15:XDP, IND:VA64:XDP, IND:KIR:XDP, and (5:5) IND:XDP were manufactured by heating at 150° C. and then allowing the samples to melt for 10 minutes according to the HM process. In addition, (5:2) IND:AF15 and IND:VA64 were manufactured at 150° C. using HME to function as control formulations. IND:KIR was not extrudable at these temperature conditions, and therefore was only processed by HM. The appearance of the processed XDP and HM granules is shown in FIG. 2. After processing, pure XDP remains a white powder, but when formulated into granules using by HM technique, the IND melted and adsorbed onto the XDP and exhibited a yellow color. This characteristic of IND was consistent between each of the formulations (even the formulation without polymer), which suggests that IND melting may involve an interaction between the acid group of IND and silanol group of XDP (Madieh et al., 2007), and the yellowing occurs as the IND covers the surface and is incorporated into the pores of XDP (Tanabe et al., 2012).

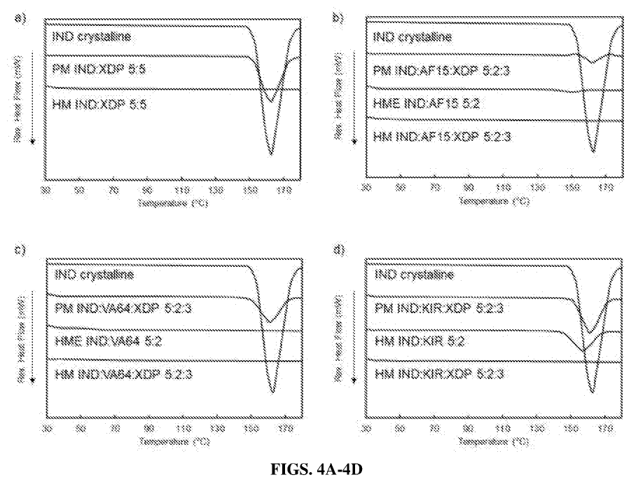

PXRD (FIG. 3) and mDSC (FIG. 4) were conducted on HM processed samples to determine amorphicity. PXRD data indicated that all samples manufactured by HM and HME process exhibited halo patterns and the absence of IND's characteristic Bragg's peaks, which indicates that the samples were amorphous. The absence of IND's melt endothermic peak on mDSC confirmed amorphicity of all processed samples containing XDP. As a control, physical mixtures (PM) were compared in order to demonstrate the benefit of thermal processing to induce amorphicity. These data demonstrate all thermally processed samples were rendered amorphous. However, for the sample not containing XDP, the crystal peak derived from IND was not observed in the IND:VA64 sample (FIG. 4C), but an endothermic peak was observed in the IND:AF15 and IND:KIR samples (FIG. 4B, 4D). We hypothesize that the endothermic peaks observed were a result of a metastable amorphous state induced by processing due to the relatively small amount of polymer compared to the amount of IND. This phenomenon was similar to that of amorphous indomethacin containing no polymer (Takeuchi et al., 2005).

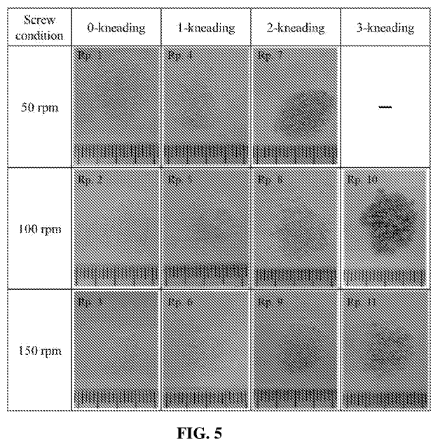

Ternary formulations were blended in a ratio of IND as drug: AF15 as polymer: XDP as MPC=5:2:3 by weight (w/w). Ternary ASDs were prepared using differing conditions by twin-screw extrusion as shown in Table 2. The appearance of all HME samples prior to milling and sieving are shown in FIG. 5. All processed samples demonstrated a color change from white to yellow due to the melting of IND. However, the sample colors of 2-kneading/50 rpm (Rp.7) and 3-kneading/100 rpm (Rp.10) were brown or dark yellow. The browning of the IND demonstrated that the samples were scorched, likely due to high shearing and high local temperature under these conditions in the HME process.

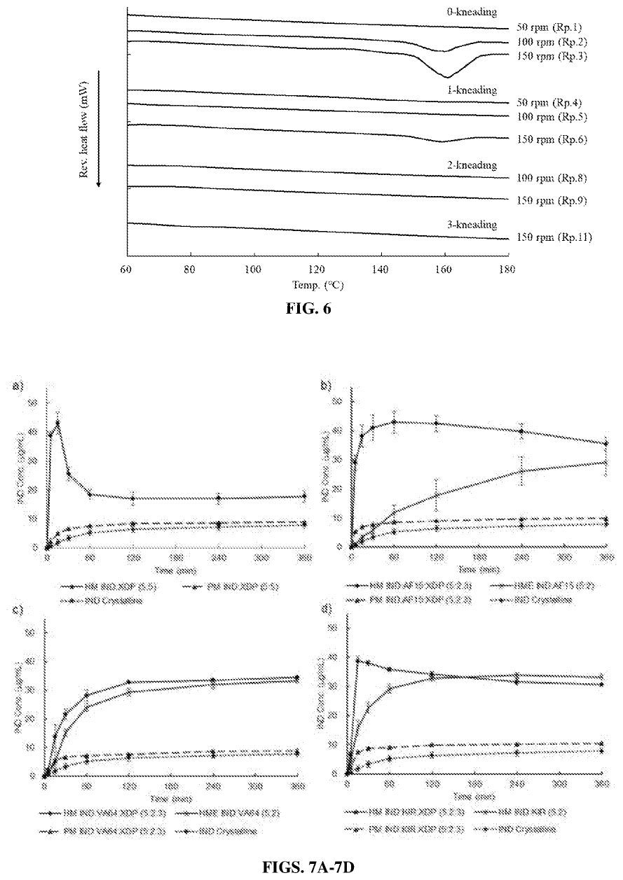

mDSC (FIG. 6) were conducted on HME processed samples to determine amorphicity. 0-kneading/100 rpm (Rp.2), 0-kneading/150 rpm (Rp.3), and 1-kneading/150 rpm (RP.6) possess an endothermic event as shown by mDSC. This indicates the respective processing conditions were unable to render IND completely amorphous, likely due to a lack of sufficient heat transfer to the samples due to the short residence time and kneading elements used in the process. For further evaluation, only completely amorphous samples (samples highlighted in the green-framed squares, FIG. 5) were evaluated. All samples were milled and sieved in order to eliminate coarse particles. As shown in Table 3, the ternary ASDs were evaluated for particle size distribution, SSA, and percent purity by HPLC. For SSA, the 0- and 1-kneading zone samples demonstrated SSA values similar to the same formulation processed via our hot melt method previously reported (21.1 m²/g) (Hanada et al. (1), 2018). The SSA of samples decreased as the number of kneading zones increased. The observed differences in SSA demonstrate that the IND/AF15 in the formulation underwent an increased local temperature at the kneading elements, leading to a subsequent decrease in the melt viscosity (liquid-like property of the material). Therefore, as the number of kneading zones increased, it became easier for the formulation to absorb into the XDP pores, leading to a lower overall SSA of the processed material. Also, Table 3 indicates that the particle size of the ternary ASDs tended to be larger than XDP. Without wishing to be bound by any theory, it is believed that the large particles are actually granules composed of discrete particles with IND/AF15 that remain on the surface of the XDP particles before being absorbed into the XDP pores, potentially due to complete saturation of the XDP pores. However, there was no correlation between particle size and SSA, and there was less influence of particle size. Quantitively, the results indicated that IND recovery after thermal processing was acceptable (see, Hanada et al, 2018 (2)).

TABLE 3
Particle size distribution, SSA, and drug recovery of ternary ASDs.

| | | Particle size distribution | | | | Drug recovery |
|---|---|---|---|---|---|---|
| Rp. | Sample | d₁₀ (μm) | d₅₀ (μm) | d₉₀ (μm) | SSA (m²/g) | (%) |
| — | XDP | 9.1 ± 0.5 | 57.3 ± 3.2 | 106.4 ± 3.4 | 286.3 ± 5.2 | — |
| — | PM | 9.0 ± 1.2 | 54.9 ± 3.3 | 136.4 ± 5.2 | 85.7 ± 0.8 | 101.8 ± 0.4 |
| 1 | 0-kneading/50 rpm | 10.6 ± 0.2 | 66.2 ± 7.5 | 178.1 ± 13.3 | 21.2 ± 0.1 | 99.6 ± 0.4 |
| 4 | 1-kneading/50 rpm | 11.3 ± 2.8 | 72.8 ± 6.5 | 195.0 ± 20.9 | 23.0 ± 0.1 | 98.3 ± 3.0 |
| 5 | 1-kneading/100 rpm | 9.6 ± 1.2 | 60.5 ± 6.7 | 164.9 ± 10.2 | 20.6 ± 0.3 | 99.2 ± 2.4 |
| 8 | 2-kneading/100 rpm | 16.4 ± 1.2 | 111.6 ± 2.4 | 244.7 ± 4.7 | 8.9 ± 0.2 | 99.4 ± 1.7 |
| 9 | 2-kneading/150 rpm | 15.7 ± 2.1 | 79.3 ± 4.6 | 189.1 ± 9.8 | 7.3 ± 0.8 | 99.8 ± 0.8 |
| 11 | 3-kneading/150 rpm | 16.0 ± 1.0 | 102.2 ± 18.1 | 243.8 ± 23.2 | 2.2 ± 0.1 | 97.4 ± 1.0 |

Each value represents the mean ± S.D. (n = 3)

### Example 3—Dissolution Behavior

Based on Example 1 and 2, the formulation compositions were characterized as follows:

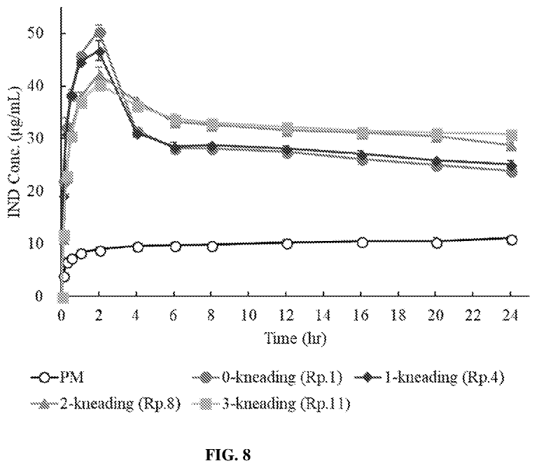

The dissolution profiles of both the HM and HME granules are compared to those of the PM and crystalline IND profiles and shown in FIG. 7. The HM granules of IND:XDP (FIG. 7A) demonstrated rapid release after the initiation of dissolution. However, precipitation and agglomeration of the granules occurred in the vessel 30 minutes after the beginning of the test, which lead to a rapid decrease in IND concentration. The rapid increase in concentration demonstrates the ability for XDP to induce supersaturation in an immediate release profile, but the absence of polymer caused the system to revert back to IND's more stable equilibrium solubility after a short period of time of less than one hour. In FIG. 7B, it is important to recognize that the HM granules containing IND:AF15:XDP also demonstrates an immediate spring to supersaturation due to the thermal processing using XDP. From this same figure, one notices the importance of presence of a polymer to inhibit precipitation and sustain the supersaturation conditions achieved by XDP processed formulations. This formulation demonstrated the “spring and parachute” effect. The HME IND:AF15 granules, on the other hand, demonstrated a dissolution rate similar to a zero-order release profile. The HME IND:AF15 granules were poorly dispersed and aggregated in the dissolution media. The IND:VA64:XDP granules (FIG. 7C) demonstrated minimal benefit of incorporating XDP in the granules, as the HME IND:VA64 dissolution curve was similar. Lastly, the IND:KIR:XDP granules (FIG. 7D) demonstrated a “spring and parachute” effect, similar to the HM IND:AF15:XDP granules. The KIR formulation lacking XDP eventually reached supersaturation, but like the AF15 formulation, it demonstrated a zero-order release profile, and the immediate release “spring” to supersaturation was not observed. Based on these data, the importance of XDP in the formulations made by HM and HME to reach supersaturation quickly and the necessity of polymer in the ternary formulation to maintain supersaturation was confirmed. However, the IND supersaturation state was dependent on the polymer employed.

Four samples (0-kneading, 1-kneading, 2-kneading and 3-kneading) were prepared using different kneading zone conditions in order to observe differences in dissolution behavior. The dissolution profiles are shown in FIG. 8. The ternary ASDs prepared by 0- and 1-kneading zones demonstrated a rapid initial dissolution and reached high supersaturation by 2 hours, and shortly thereafter, precipitation of the samples occurred before any parachute effect was observed to maintain supersaturation. A parachute effect occurred at concentrations of about 3× the equilibrium solubility of crystalline IND. Meanwhile, the ASDs prepared by 2- and 3-kneading zones also demonstrated rapid dissolution to about 4× equilibrium solubility of IND up to 2 hours, but the resulting precipitation phase was less significant and the resulting parachute demonstrated an ability to maintain higher dissolution rates throughout the remainder of the 24-hour time period when compared to those exhibited by samples processed using the conditions of smaller number of kneading elements. In order to investigate the observed differences in the dissolution profiles between the samples processed by differing the number of kneading elements, SME values, C_{max-disol}, and AUDC₀₋₂₄ hr were calculated (Table 4). The calculated SME values were similar for the 0- and 1-kneading element samples, whereas 2- and 3-kneading samples demonstrated about 20- and 40-times higher values than the 0- and 1-kneading samples, respectively. In addition, C_{max-dissol} showed higher values as the number of kneading zones was decreased, while AUDC₀₋₂₄ hr showed a tendency to increase as the number of kneading zones was increased. From these results, two dissolution profile patterns can be used to categorize our samples as low-SME samples (0- and 1-kneading) and high-SME samples (2- and 3-kneading). Without wishing to be bound by any theory, it is believed that the difference in dissolution behavior (i.e., parachute effect) of the samples is a result of different levels of interaction between our API-polymer blend and XDP, which results in different recrystallization behavior in solution. Yani et al report that the lack of interaction between fenofibrate and PVP64 led to poor stability of the fenofibrate/PVP64 solid dispersion, as evident from recrystallization of fenofibrate from the solid dispersion during storage and the characteristic parachute dissolution behavior of the solid dispersion (Yani et al., 2017). Furthermore, Chauhan et al report that a correlation is observed in the polymer's ability to inhibit precipitation in solution and ability to maintain amorphicity in the solid state with indomethacin. They explain that this phenomenon can be understood by the strength of drug-polymer interactions (Chauhan et al., 2014). Thus, we assumed that SME can influence the absorption behavior of IND/AF15 into XDP pores or can have an effect on the miscibility and interactions between IND and AF15 (see, Hanada et al, 2018 (2)).

TABLE 4
SME, C_{max-dissol,} and AUDC of ternary ASDs.

| | | | SME**(kW · | C_{max-dissol} | AUDC_{0-24 hr}***(μg · |
|---|---|---|---|---|---|
| Rp. | Sample | Torque*(Gm) | hr/kg) | (μg/mL) | hr/mL) |
| — | PM | — | — | 11.0 ± 0.3 | 239.1 ± 9.5 |
| 1 | 0-kneading | 5 | 0.005 | 50.5 ± 1.2 | 700.3 ± 2.9 |
| 4 | 1-kneading | 6 | 0.005 | 46.7 ± 1.9 | 706.6 ± 4.4 |
| 8 | 2-kneading | 50 | 0.092 | 42.2 ± 1.4 | 778.0 ± 5.2 |
| 11 | 3-kneading | 70 | 0.190 | 40.4 ± 0.5 | 786.6 ± 7.5 |

*Torque was averaged from 2 to 5 min in process
**SME was calculated by and Eq. (3) and (4).
***Calculated by using the linear trapezoidal method

### Example 4—Investigating Differences in Dissolution Profiles of Ternary Formulations—Rheological Differences

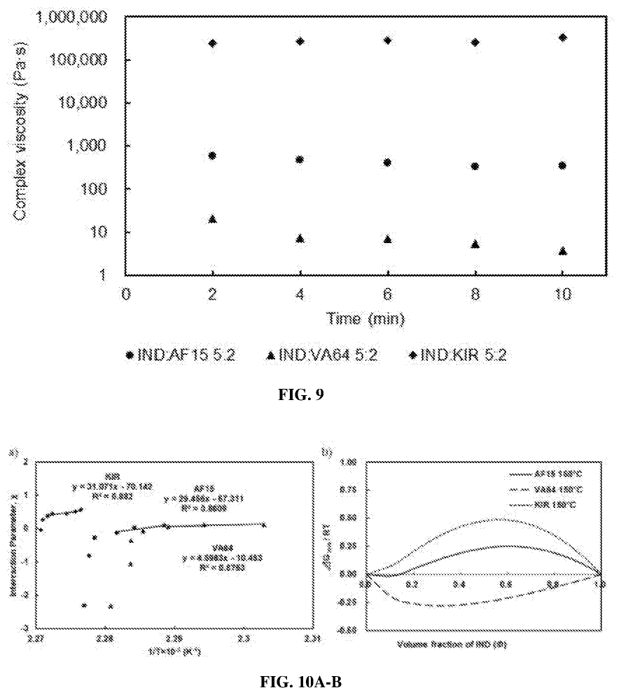

During evaluation of the dissolution studies, it was noted that the observed benefits of XDP in the formulation only occurred with the AF15 and KIR formulations, and not the VA64 formulations. We also observed how the differing viscosities between the polymers can have an effect on the adsorption onto the XDP particles and eventually affect the dissolution rate. To evaluate the viscosity differences during our HM process, rheology was performed on drug:polymer blends in a ratio of 5:2 and purposely excluded XDP due to its inability to melt during processing. To simulate the HM process in the rheometer, the testing conditions were carried out at 150° C. for 10 minutes and the complex viscosity was evaluated. Complex viscosity (η, Pa·s) is calculated from Storage modulus (G′, Pa) and Loss modulus (G″, Pa), and was used to understand the viscosity differences in the samples as a function of time while at the processing temperature. The rheology data (FIG. 9) demonstrated a wide range in complex viscosities between the three polymers. At 10 minutes IND:VA64 had the lowest complex viscosity (3.7 η, Pa·s), whereas IND:AF15 had a higher complex viscosity (341.3 Pa·s), and IND:KIR had the highest complex viscosity (327,856.6 Pa·s). The low complex viscosity observed in the IND:VA64 sample was 100× and 100,000× lower than the observed complex viscosities of the IND:AF15 and IND:KIR samples, respectively. It is hypothesized that the significantly lower complex viscosity of the VA64 sample leads to more efficient spreading of drug and polymer over and into the pores of XDP during manufacturing, in turn causing slower initial dissolution.

### Example 5—Miscibility of IND and Each Polymer Based on Flory-Huggins Theory

Next, miscibility of IND and each polymer was investigated based on Flory-Huggins theory. The Flory-Huggins interaction parameter, χ, was calculated according to Equation 3, using Table 5 values and the end melting point temperature of IND observed during DSC. For the IND:AF15, a linear relationship between 1/T and χ was observed across the experimental formulation range from 0.75 to 0.65. The IND:VA64 and IND:KIR samples showed a linear relationship at the formulation range from 0.70 to 0.60 and 0.80 to 0.60, respectively. At drug concentrations higher than 75% w/w (AF15), 75% w/w (VA64), and 80% w/w (KIR), a nonlinear relationship was observed. Nonlinearity between 1/T and χ has been previously reported at high drug loading of IND in a PVP-VA formulation (Zhao et al., 2011; Tian et al., 2013). This phenomenon is explained by specific drug-polymer blends, as the interaction parameter may be dependent upon higher order concentration terms. One point to consider is that nonlinearity occurs at high drug loadings and small values of 1/T, which generally occurs at higher temperatures (Tian et al., 2013).

TABLE 5
IND and polymer properties used for
Flory-Huggins theory modeling.

| | MW | Density | Molecular volume* | ΔH_{fus} |
|---|---|---|---|---|
| | (g/mol) | (g/cm³) | (cm³/mol) | (kJ/mol) |
| IND | 357.79 | 1.38⁽ⁱ⁾ | 259.27 | 37.32** |
| AF15 | 85,000 | 1.20⁽ⁱⁱ⁾ | 70833.33 | — |
| VA64 | 57,500 | 0.97⁽ⁱⁱⁱ⁾ | 59278.35 | — |
| KIR | 45,000 | 1.15^{(iv)} | 39130.43 | — |

*Values calculated by dividing molecular weight by true density;
**Calculated from DSC
⁽ⁱ⁾Xiang and Anderson, 2013.
⁽ⁱⁱ⁾Affmisol ™ HPMC HME for hot melt extrusion, D.P.F. Solutions, Editor.
⁽ⁱⁱⁱ⁾Altamimi and Neau, 2016.
^{(iv)}Suhrenbrock et al., 2011.

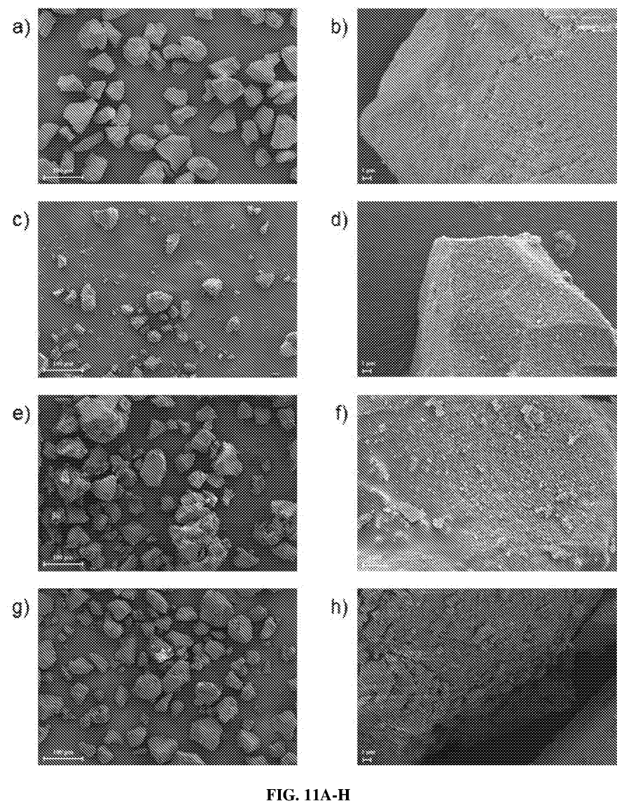

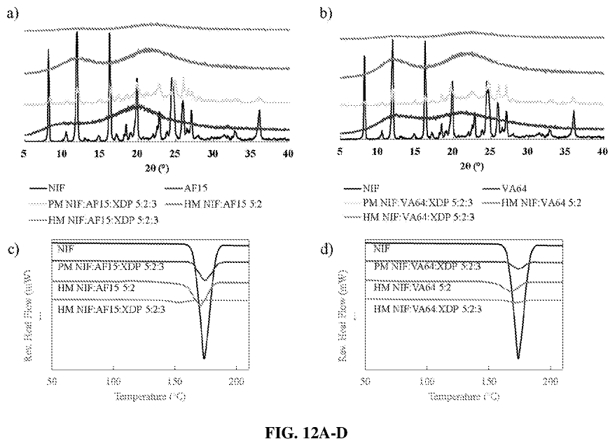

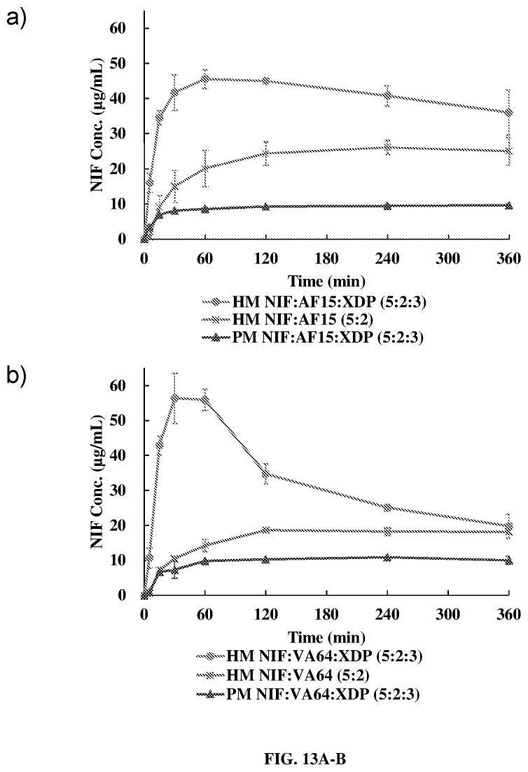

The Gibb's free energy of mixing (ΔGₘᵢₓ) as a function of drug composition and 150° C. was calculated according to Equation 1, using the slope and intercept values derived from 1/T and χ. The χ, values for AF15, VA64, and KIR were 2.32, 0.41, and 3.31, respectively. As a result, AF15 and KIR indicated positive ΔGₘᵢₓ values, while the ΔGₘᵢₓ for VA64 was negative. The negative ΔGₘᵢₓ for VA64 indicated higher miscibility with IND than either AF15 or KIR (FIG. 10).

Based on the rheology data and Flory-Huggins modeling, the dissolution rate of IND contained in VA64 granules would likely not benefit from including XDP in the drug-polymer mixture made by a thermal process or fusion-based high energy process, as compared to AF15 and KIR polymers. Based on Flory-Huggins theory, IND and VA64 exhibited miscibility that suggests stability and miscibility of the amorphous system during dissolution. As this drug-polymer mixture exhibited dissolution enhancement, the observed benefit from the HM processed granules containing XDP was minimal. Therefore, the IND:VA64 granules are a positive control to compare the benefits observed of XDP-containing granules with drug-polymer formulations that demonstrated lower miscibility by Flory-Huggins theory modeling.

The results indicate that the increased miscibility of the IND:VA64 formulation and the substantially lower viscosity of the granules also led to increased surface coverage onto the XDP particles as compared to the AF15 and KIR granules. To further study this proposed mechanism, the specific surface area (SSA) of the HM granules by BET (Table 6) was determined. The IND:VA64:XDP exhibited a substantially lower surface area compared to the other formulations containing XDP. Also, the SSA of the ternary mixtures directly correlated with the results of the complex viscosity and drug-polymer miscibility based on Flory-Huggins theory. It is postulated that the lower surface area indicates more coverage onto the surface of the XDP particles and blockage of the pores. The data demonstrated that the IND:VA64 blend had the lowest complex viscosity (meaning it is most likely to flow into and block the pores of the XDP during thermal processing) and the most negative ΔG_{MIX}, which further indicates the best miscibility between the IND and polymer, based on the polymers studied. SEM was used (FIG. 11) to visualize the surface of the HM XDP-containing granules with each polymer, expecting more visible coverage between the IND:VA64:XDP granules compared to the AF15 and KIR containing formulations. SEM confirmed visually greater surface coverage of the IND:VA64 particles onto XDP since the SEM picture exhibits a film-like membrane of IND:VA64 onto XDP (FIG. 11F). For the IND:KIR:XDP granules, IND- and KIR-rich domains on the spherical particles were observed. The inconsistent surface coverage of the particles was secondary to the complex viscosity of KIR being too high at 150° C. (FIG. 11G).

TABLE 6
BET analysis of samples containing XDP

| Formulation | BET (m²/g) |
|---|---|
| XDP | 316.0 |
| HM IND:XDP 5:5 | 88.2 |
| HM IND:AF15:XDP 5:2:3 | 21.1 |
| HM IND:VA64:XDP 5:2:3 | 10.6 |
| HM IND:KIR:XDP 5:2:3 | 21.4 |
| HME IND:AF15 5:2 | 0.3 |
| HME IND:VA64 5:2 | 0.1 |
| HM IND:KIR 5:2 | 0.5 |

The results demonstrate the ability to manufacture an ASD employing a thermal process without the use of a solvent. The results also demonstrate the ability to predict which drug-polymer formulation will benefit in terms of dissolution rate from HM-processed XDP by using Flory-Huggins theory in pre-formulation assessment. Drug-polymer formulations that demonstrate high miscibility may not benefit from the HM or HME process that incorporates XDP. Though other researchers have reported the ability to prepare ASDs using mesoporous carriers, their methods required heating the drug to its melting point or imparting high shear forces (Hoashi et al., 2011; Maniruzzaman et al., 2015; Nakahashi et al., 2014; Shibata et al., 2009; Fujii et al., 2005). The present disclosure teaches that an ASD can be prepared at temperatures below the drug's melting point without using mechanical stress by incorporating polymer and MPS in the formulation. From these results, HM and HME processes can be employed to not cause the chemical degradation of drug due to lower heat required during manufacturing. Without wishing to be bound by any theory, it is believed that successful enhancement of dissolution properties of the drug by using XDP in a ternary mixture of drug and polymer appears to correlate with the following properties of the composition: 1) Low miscibility of polymer with drug; 2) Melting point depression between drug and polymer as a result of drug interaction with the silanol group on XDP (Nakagami, 1991) making it possible to manufacture below the melting point of IND, which minimizes IND chemical degradation; and 3) Complex viscosity of polymer is not too low, which causes formation of a film that covers the surface of XDP and blocks the pores of the XDP particles, thus inhibiting the potential benefits of having exposed silanol groups on the surface of the HM granules.

MPS contains many hydrophilic silanol groups on the surface of the particles, which improve the wettability of the system. Therefore, larger SSAs maintained after thermal processing resulted in greater observed initial dissolution rates. In order to ensure that HM processed formulations retain the high SSA characteristic of MPS, appropriate selection of polymer is important for successfully achieving of the “spring and parachute” effect during drug dissolution. The results demonstrate the ability to maintain a higher SSA after thermal processing by selecting a polymer with lower drug-miscibility and higher complex viscosity. For solid dispersion systems employing MPS with polymer(s), these results indicate that it is possible to guide the formulation effort to achieve an immediate spring in drug release and maintenance of drug supersaturation by investigating the miscibility and complex viscosity of the drug-polymer composition.

### Example 6—Miscibility of IND and AF15 in Ternary ASDs that were Prepared by Nano-16 Based on Evaluating ssNMR

ssNMR was utilized to analyze miscibility, phase structure, and heterogeneity in drug-polymer mixtures on a molecular scale (Ukmar et al., 2012; Vogt et al., 2013; Yuan et al., 2014; Yang et al., 2016; Purohit et al., 2017). To examine the nature of the crystalline and amorphous components in different samples, ¹³C cross-polarization magic angle spinning (CP-MAS) spectra of ternary ASDs, IND (crystal, amorphous), AF15 and PM (containing IND crystal) are acquired. All ¹³C signals in each spectrum were assigned to IND and/or AF15 molecules, as XDP did not show detectable carbon intensity. These well-resolved ¹³C MAS NMR spectra provide an opportunity to measure the individual ¹H relaxation behaviors in the laboratory and rotating frame, respectively (Stejskal et al, 1981 Wu et al., 2002, Yang et al., 2016; Purohit et al., 2017) in the ¹³C-detected manner. ¹H-NMR spin-lattice relaxation measurements were shown to be useful for assessing the miscibility and quantifying phase-separated domain size of a drug and excipients in ASDs prepared by different composition ratios and methods when the Tg is not clearly detected by DSC (Aso et al., 2007; Yuan et al., 2014; Yang et al., 2016; Purohit et al., 2017). The NMR relaxation values of each components in ASDs usually reflects the averaged property of multiple nearby nuclei due to homonuclear spin diffusion occurring during the dipolar-coupling-based cross polarization. Based on the measured ¹H spin-lattice relaxation time in the laboratory frame (T₁) and rotating frame (T_{1ρ}), miscibility between API and polymer was evaluated following three classifications: (i) Miscible, both T₁ and T_{1ρ} values will be same for API and polymer; (ii) Partly miscible, the T_{1ρ} values will be different for API and polymer but the T₁ values will be the same; (iii) Immiscible, both T₁ and T_{1ρ} values will be different for API and polymer. The magnetization of both IND and AF15 grows exponentially at different rates, indicating distinct relaxation times for the two components. The values of T₁ and T_{1ρ} relaxations were derived via curve fitting using the two equations provided in the ssNMR method session, respectively, and demonstrated along with the build-up curves. T₁ and T_{1ρ} relaxation times can provide estimates of the diffusive path length and the sizes of blend heterogeneities. A practical approximate estimation of the upper limit to the domain size can be obtained (Wu et al., 2002). Briefly, it can be calculated by the following equation (Wu et al., 2002. Aso et al., 200T Yuan et al., 2014, Clauss et al., 1993. Purohit et al., 2017);

L=\sqrt{6Dt} (Equation 8)

where L is magnetization diffusion across a length and describes the domain size. D is the spin diffusion coefficient and t is the relaxation time. The diffusion coefficient of organic polymers is 8.0×10−12 cm²/s for a rigid system (Wu et al., 2002; Clauss et al., 1993; Purohit et al., 2017; Brettmann et al., 2012). For major pharmaceutical components, T₁ and T_{1ρ} are on the order of 1 s and 10 ms, respectively, and can characterize differing domain sizes at the length scale of 20-100 nm and 1-20 nm, respectively (Purohit et al., 2017). The relaxation results suggested an interesting correlation between IND/AF15 miscibility and the different HME processes. Table 7 summarizes the average values of measured relaxation times and corresponding standard error bars. Their domain sizes were estimated using Eq. (8) and shown in the table. For the 0- and 1-kneading samples, T₁ and T_{1ρ} values between IND and AF15 were distinct, indicating that IND and AF15 were distributed in different domain sizes from about 1 to 100 nm length scales. Thus, the 0- and 1-kneading samples were determined to be immiscible. For the 2- and 3-kneading samples, T₁ values of IND and AF15 were identical but T_{1ρ} values were different. This indicated that IND and AF15 were miscible around ca 85-100 nm length scale but immiscible around ca 7-10 nm length scale. Consequently, the 2- and 3-kneading samples were determined to be partly miscible. From these results, none of the processed ternary ASDs were considered to be miscible. However, the ΔT₁ and ΔT_{1ρ} values, revealed the differences in relaxation times between IND and AF15 for each sample tended to decrease with an increasing number of kneading zones. The data suggest that dedicated kneading zones improved IND/AF15 miscibility, especially at the length scale of 20-100 nm, where 2- and 3-kneading samples led to partially miscible ternary-ASDs with zero ΔT₁. At the same time, the smaller ΔT_{1ρ} values between IND and AF15 in ternary ASDs from 10.8 ms to 3.0 ms with high-SME inputs indicated the API and polymer formed more miscible systems at a molecular level. For the low-SME case (i.e. 0-kneading), IND and AF15 are apart from each other at more than 100 nm molecular distance, significantly different relaxation times show the two components are immiscible in the ternary ASD. In the case of high-SME mixing (i.e., 3-kneading), IND and AF15 share efficient spin diffusion between intermolecular protons due to their proximities at ca 20-100 nm domain size. By improving the IND/AF15 phase separation in smaller length scale of 1-20 nm, more kneading zones with high-SME may be necessary to achieve fully miscible ternary ASD samples. It is interesting to observe the relatively smaller relaxation values of Rp.11 than other ASDs in Table 7 (see, Hanada et al, 2018 (2)).

TABLE 7
IND-AF15 miscibility evaluated from ¹H spin-lattice relaxation measurements.

| | | | ΔT₁ | Domain | | ΔT_{1ρ} | Domain | |
|---|---|---|---|---|---|---|---|---|
| | | T₁ | (s) | size (nm) | T_{1 ρ} | (ms) | size (nm) | Miscibility |
| Samples | | | | | | | | |
| 0-kneading (Rp.1) | IND | 2.2 +0.1 | 0.3 | 103 | 24.0 ± 0.5 | 10.8 | 10.7 | Immiscible |
| SME: 0.005 kW · | AF15 | 1.9 ± 0.1 | | 95 | 13.2 ± 1.0 | | 8.0 | |
| hr/kg | | | | | | | | |
| 1-kneading (Rp.4) | IND | 2.1 ± 0.1 | 0.3 | 100 | 21.0 ± 0.5 | 8.0 | 10.0 | Immiscible |
| SME: 0.005 kW · | AF15 | 1.8 ± 0.1 | | 93 | 13.0 ± 0.5 | | 7.9 | |
| hr/kg | | | | | | | | |
| 2-kneading (Rp.8) | IND | 2.2 ± 0.1 | 0 | 103 | 20.2 ± 0.3 | 6.2 | 9.8 | Partly |
| SME: 0.092 kW · | AF15 | 2.2 ± 0.2 | | 103 | 14.0 ± 0.2 | | 8.2 | Miscible |
| hr/kg | | | | | | | | |
| 3-kneading (Rp.11) | IND | 1.5 ± 0.1 | 0 | 85 | 13.0 ± 1.0 | 3.0 | 7.9 | Partly |
| SME: 0.190 kW · | AF15 | 1.5 ± 0.1 | | 85 | 10.0 ± 0.5 | | 6.9 | Miscible |
| hr/kg | | | | | | | | |
| Reference samples | | | | | | | | |
| Amorphous IND | IND | 2.4 ± 0.2 | — | 107 | 24.0 ± 3.0 | — | 10.7 | — |
| AF15 | HPMC | 1.7 ± 0.2 | — | 90 | 13.0 ± 2.0 | — | 7.9 | — |

### Example 7—Recrystallization Behavior of ASD Samples

The ternary ASD samples were stored at elevated stability conditions of 40° C. and 75% RH for up to 14 days (Table 8). At Day 1, the 3-kneading sample did not exhibit IND crystalline peaks, while all other samples showed recrystallization, which was observed by PXRD. Samples prepared with increasing number of kneading zones tended to suppress the tendency to recrystallize up through Day 3. Both PXRD and mDSC results showed all samples possessed similar levels of crystallinity after Day 7. By PXRD, some recrystallization peaks (approximately 9° and 15°) were different from the peaks pattern of PM. These peaks were quite similar to the α-form of IND. It has been reported that the α-form generated from amorphous IND changed with storage temperature (Yoshioka et al., 1994; Ueda et al., 2014. Kaneniwa et al., 1985). Using mDSC, the endothermal event shifted to around 130° C. rather than 160° C. found in the PM. However, this melting point shift was not derived from any pure crystal form because the α and γ crystal melting points are 155° C. and 161° C., respectively (Yoshioka et al., 1994) and because PXRD results exhibited the characteristic diffraction pattern of the γ crystal from. This discrepancy may be explained by the following; (i) there was a melting point depression effect with polymer, or (ii) the porous material effect, where it was reported that high API concentration in mixtures demonstrated a broad endothermic peak at a lower temperature than the melting point (Nakai et al., 1984; Matsumoto et al., 1998). As the samples are exposed to elevated temperature and moisture, crystal nuclei generate from an active IND-rich phase. From the stability test data, the samples with low IND-AF15 miscibility possess IND-rich phases, or the rich phases are formed by IND molecules moving easily, because IND molecular movement is increased during exposure to moisture. In addition, high-SME samples demonstrated delayed recrystallization, because the viscosity of IND and AF15 solution decreased and facilitated the absorption of the IND/AF15 blend into the pores of XDP. More efficient absorption into the XDP pores then protected IND from recrystallization when the blend was exposed to high temperature and humidity conditions. The crystallinity behavior trended similarly with the PXRD and DSC results. The recrystallization of IND may be occurring on the XDP surface, because PXRD relies on the principle of measuring the sample surface for the determination of crystallinity From storage days 7-14, the crystallinity of all samples demonstrated no differences from IND crystal diffraction intensity on PXRD or IND crystal endothermal events on mDSC. The tendency to recrystallize must be understood as it is affected by processing, where high-SME samples demonstrated a slower rate of recrystallization. From these results, recrystallization rate of drug can be suppressed by increasing SME by increasing the number of kneading zones. A better miscibility produced at a higher SME input, as identified and quantified by ssNMR relaxation measurements, promote the physical stability of the IND/AF15 ASDs (see, Hanada et al, 2018 (2)).

TABLE 8
Crystallinity of ternary ASDs prepared by different SME.

| Storage term | | Day 1 | Day 3 | Day 7 | Day 14 |
|---|---|---|---|---|---|
| 0-kneading | PXRD (n = 3) | 16.1 ± 1.1 | 27.3 ± 3.6 | 24.1 ± 6.3 | 25.6 ± 5.7 |
| (Rp.1) | mDSC (n = 1) | 15.3 | 25.2 | 21.8 | 23.1 |
| 1-kneading | PXRD (n = 3) | 8.5 ± 1.9 | 20.8 ± 8.6 | 26.9 ± 3.3 | 31.3 ± 3.6 |
| (Rp.4) | mDSC (n = 1) | 7.3 | 27.0 | 23.4 | 21.8 |
| 2-kneading | PXRD (n = 3) | 3.5 ± 0.5 | 14.5 ± 2.4 | 24.1 ± 3.2 | 22.9 ± 4.6 |
| (Rp.8) | mDSC (n = 1) | 3.6 | 16.8 | 25.0 | 21.8 |
| 3-kneading | PXRD (n = 3) | 0.0 ± 0.0 | 9.6 ± 0.3 | 24.8 ± 5.3 | 26.3 ± 4.8 |
| (Rp.11) | mDSC (n = 1) | 3.3 | 10.8 | 23.9 | 23.3 |

### Example 8—Preparation and Properties of Formulations Comprising Nifedipine

In other embodiments, the formulations comprise nifedipine. Physicochemical properties of nifedipine are illustrated in Table 9.

TABLE 9
Physicochemical properties of Nifedipine.

| | |
|---|---|
| API | Nifedipine |
| Structure | 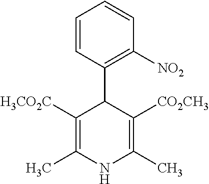 [SMILES: COC(=O)C1=C(C)NC(C)=C(C(=O)OC)C1c1ccccc1[N+](=O)[O-]] |
| Melting | 172-174° C. |
| point | |
| Solubility | 10 μg/mL (pH 1.2) |
| (37° C.) | 10 μg/mL (pH 4.0) |
| | 10 μg/mL (pH 6.8) |
| | 10 μg/mL (water) |
| Stability | 37° C., 24 hr under light shielding |
| (water) | |
| Stability | Degradation more than 95% at |
| (light) | 26,700 lx · hr |

TABLE 10
Formulations made by thermal processing comprising nifedipine

| | Thermal | | | | | Ratio |
|---|---|---|---|---|---|---|
| | Processing | NIF | AF15 | VA64 | XDP | (NIF:Polymer: |
| Formulation | Technique | (%) | (%) | (%) | (%) | XDP) |
| 8 | HM | 50 | 20 | — | 30 | 5:2:3 |
| 9 | HM | 50 | — | 20 | 30 | 5:2:3 |
| 10 | HM | 71.4 | 28.6 | — | — | 5:2:0 |
| 11 | HM | 71.4 | — | 28.6 | — | 5:2:0 |

HM: Hot Melt Granulation; NIF: Nifedipine; AF15: Hypromellose (Affinisol HPMC HME 15LV); VA64: Copovidone (Kollidon VA64); XDP: Hydrous silicon dioxide (Syloid XDP 3050)

The ingredients of formulations 8-11 as described in Table 10 were mixed until uniform using a mortar and pestle, and then each composition was heated at 165° C. for 15 minutes in a Breville Smart Oven® Pro (Breville USA, Torrance Calif.). The compositions were removed from the hot melt granulating step and allowed to cool to room temperature (about 25° C.). Formulations 8 and 9 (see Table 10) were sized using a mortar and pestle such that the granules passed through a 212 μm mesh screen. Formulations 10 and 11 (see Table 10) were sized using a mechanical milling machine (e.g., a grinder) to form granules that passed through a 212 μm mesh screen.

A Hanson SR8PLUS dissolution test apparatus 2 (Hanson Research Co., Chatsworth, Calif.) (paddles) was used to perform dissolution testing. The paddle speed and temperature were set to 100 rpm and 37° C.±0.5° C., respectively. Deionized water (900 mL) was pre-heated to 37° C. in each dissolution vessel. Aliquots of granules from formulations 8-11 containing about 200 mg NIF equivalent (n=3) were then added immediately to the dissolution vessel. 2 mL samples of the dissolution media were withdrawn at 15 min, 30 min, 1 h, 2 h, 4 h and 6 h, and filtered through a 0.45-μm 25-mm PES membrane filter. A 500 μL aliquot of the filtered solution was then diluted with 500 μL HPLC grade acetonitrile, and the concentration of NIF in the diluted sample was determined using HPLC. See FIGS. 13A-B.

PXRD studies were conducted on a Rigaku Miniflex600 II (Rigaku Americas, The Woodlands, Tex.) instrument equipped with a Cu-Kα radiation source generated at 40 kV and 15 mA. The 2-theta angle, step size, and scan speed were set to 5°-40°, 0.02°, and 5°, respectively. In order to obtain PXRD patterns, the raw data was processed using PDXL2 software (Rigaku Americas, The Woodlands, Tex.). See FIGS. 12A-B.

mDSC equipped with a DSC refrigerated cooling system (DSC 2920, TA Instruments, New Castle, Del.) was employed. Dry nitrogen gas at a flow rate of 40 mL/min throughout the testing was used to purge the DSC cell. Samples were accurately weighed in aluminum sample pan kits (PerkinElmer Inc., Shelton, Conn.) and crimped before analysis. Samples were heated from 25° C. to 250° C. with a heating ramp rate of 10° C./min using a 1° C./60 sec modulation program. TA Universal Analysis 2000 software was used to process the raw data. See FIGS. 12C-D.

### Example 9—Preparation and Properties of Formulations Comprising Ritonavir

In other embodiments, the formulations comprise ritonavir. Ritonavir is a poorly water-soluble drug.

The ingredients of formulations 12 and 13 as described in Table 11 were mixed until uniform using a mortar and pestle, and then each composition was heated at 115° C. for 10 minutes in a Breville Smart Oven® Pro (Breville USA, Torrance Calif.). The compositions were removed from the hot melt granulating step and allowed to cool to room temperature (about 25° C.). Formulations 12 (see Table 11) were sized using a mortar and pestle such that the granules passed through a 212 μm mesh screen. Formulations 13 (see Table 11) were sized using a mechanical milling machine (e.g., a grinder) to form granules that passed through a 212 μm mesh screen.

TABLE 11
Formulations made by thermal processing comprising ritonavir.

| | Thermal | | | | |
|---|---|---|---|---|---|
| | Processing | RTV | VA64 | XDP | Ratio |
| Formulation | Technique | (%) | (%) | (%) | (RTV:Polymer:XDP) |
| 12 | HM | 50 | 20 | 30 | 5:2:3 |
| 13 | HM | 71.4 | 28.6 | — | 5:2:0 |

HM: Hot Melt Granulation; RTV: Ritonavir; VA64: Copovidone (Kollidon VA64); XDP: Hydrous silicon dioxide (Syloid XDP 3050)

A Hanson SR8PLUS dissolution test apparatus 2 (Hanson Research Co., Chatsworth, Calif.) (paddles) was used to perform dissolution testing. The paddle speed and temperature were set to 100 rpm and 37° C.±0.5° C., respectively. Deionized water (900 mL) was pre-heated to 37° C. in each dissolution vessel. Aliquots of granules from formulations 12 and 13 containing about 200 mg RTV equivalent (n=3) were then added immediately to the dissolution vessel. 2 mL samples of the dissolution media were withdrawn at 5 min, 15 min, 30 min, 1 h, 2 h, 4 h and 6 h, and filtered through a 0.45-μm 25-mm PES membrane filter. A 500 μL aliquot of the filtered solution was then diluted with 500 μL HPLC grade acetonitrile, and the concentration of RTV in the diluted sample was determined using HPLC. See FIG. 15.

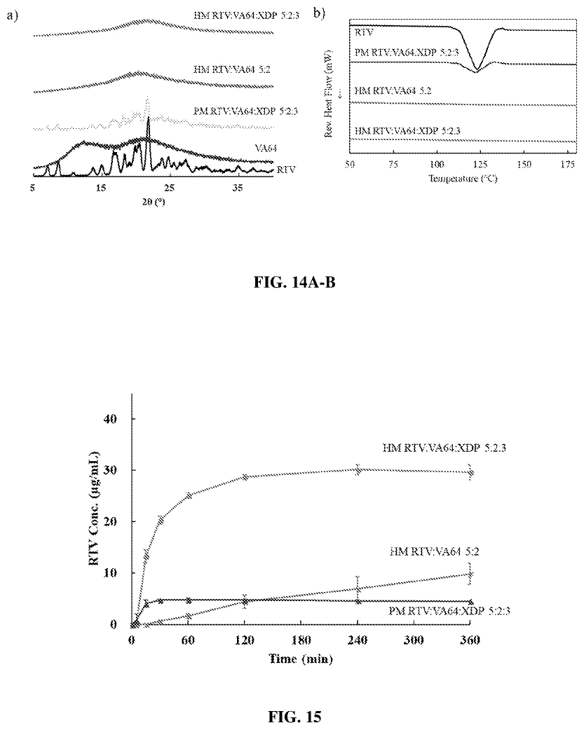

PXRD studies were conducted on a Rigaku Miniflex600 II (Rigaku Americas, The Woodlands, Tex.) instrument equipped with a Cu-Kα radiation source generated at 40 kV and 15 mA. The 2-theta angle, step size, and scan speed were set to 5°-40°, 0.02°, and 5°/min, respectively. In order to obtain PXRD patterns, the raw data was processed using PDXL2 software (Rigaku Americas, The Woodlands, Tex.). See FIG. 14A.

mDSC equipped with a DSC refrigerated cooling system (DSC 2920, TA Instruments, New Castle, Del.) was employed. Dry nitrogen gas at a flow rate of 40 mL/min throughout the testing was used to purge the DSC cell. Samples were accurately weighed in aluminum sample pan kits (PerkinElmer Inc., Shelton, Conn.) and crimped before analysis. Samples were heated from 25° C. to 200° C. with a heating ramp rate of 10° C./min using a 1° C./60 sec modulation program. TA Universal Analysis 2000 software was used to process the raw data. See FIG. 14B.

### Example 10—Effect of XDP on Itraconazole (ITZ) Dissolution at a pH where ITZ is Insoluble

In other embodiments, the formulations comprise ITZ. An ASD of ITZ was prepared with and without XDP by HM method. The ingredients of formulations 14 and 15 as described in Table 12 were mixed until uniform using a mortar and pestle, and then each composition was heated at 165° C. for 5 minutes in a Breville Smart Oven® Pro (Breville USA, Torrance Calif.). The compositions were removed from the hot melt granulating step and allowed to cool to room temperature (about 25° C.). Formulations 14 (see Table 12) were sized using a mortar and pestle such that the granules passed through a 212 μm mesh screen. Formulations 15 (see Table 12) were sized using a mechanical milling machine (e.g., a grinder) to form granules that passed through a 212 μm mesh screen.

TABLE 12
Formulations made by thermal processing comprising ITZ.

| | Thermal | | | | |
|---|---|---|---|---|---|
| | Processing | ITZ | AF4M | XDP | Ratio |
| Formulation | Technique | (%) | (%) | (%) | (ITZ:Polymer:XDP) |
| 14 | HM | 50 | 20 | 30 | 5:2:3 |
| 15 | HM | 71.4 | 28.6 | — | 5:2:0 |

HM: Hot Melt Granulation; ITZ: Itraconazole; AF4M: Hypromellose (Affinisol HPMC HME 4M); XDP: Hydrous silicon dioxide (Syloid XDP 3050)

A Hanson SR8PLUS dissolution test apparatus 2 (Hanson Research Co., Chatsworth, Calif.) (paddles) was used to perform dissolution testing. The paddle speed and temperature were set to 100 rpm and 37° C.±0.5° C., respectively. pH 6.8 phosphate buffer (900 mL) was pre-heated to 37° C. in each dissolution vessel. Aliquots of granules from formulations 14, 15 and PM (formulation 14) containing about 200 mg ITZ equivalent (n=3) were then added immediately to the dissolution vessel. 2 mL samples of the dissolution media were withdrawn at 5 min, 15 min, 30 min, 1 h, 2 h, 4 h and 6 h, and filtered through a 0.45-μm 25-mm PES membrane filter. A 500 μL aliquot of the filtered solution was then diluted with 500 μL HPLC grade acetonitrile, and the concentration of ITZ in the diluted sample was determined using HPLC. See FIG. 17.

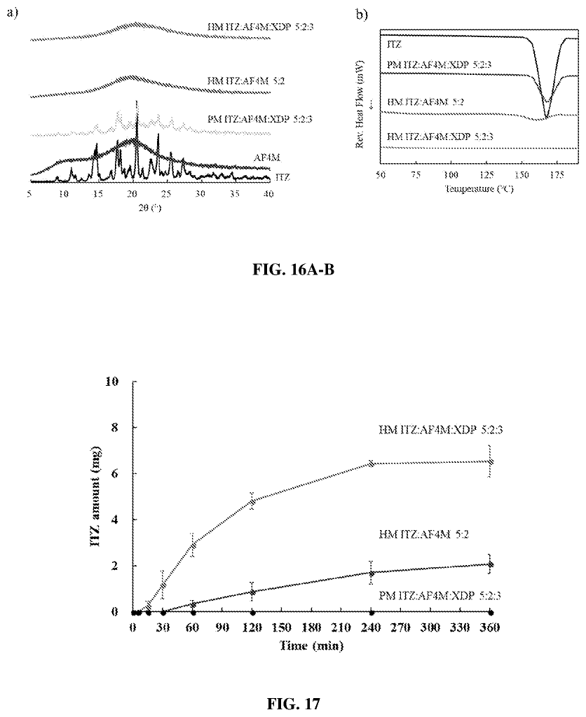

PXRD studies were conducted on a Rigaku Miniflex600 II (Rigaku Americas, The Woodlands, Tex.) instrument equipped with a Cu-Kα radiation source generated at 40 kV and 15 mA. The 2-theta angle, step size, and scan speed were set to 5°-40°, 0.02°, and 5°/min, respectively. In order to obtain PXRD patterns, the raw data was processed using PDXL2 software (Rigaku Americas, The Woodlands, Tex.). See FIG. 16A.

mDSC equipped with a DSC refrigerated cooling system (DSC 2920, TA Instruments, New Castle, Del.) was employed. Dry nitrogen gas at a flow rate of 40 mL/min throughout the testing was used to purge the DSC cell. Samples were accurately weighed in aluminum sample pan kits (PerkinElmer Inc., Shelton, Conn.) and crimped before analysis. Samples were heated from 25° C. to 220° C. with a heating ramp rate of 10° C./min using a 1° C./60 sec modulation program. TA Universal Analysis 2000 software was used to process the raw data. See FIG. 16B.

### Example 11—Effect of HME Process on ITZ Dissolution at a pH where ITZ is Insoluble

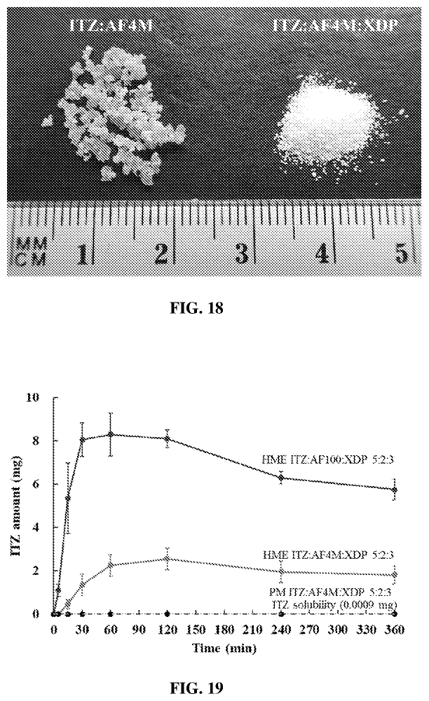

An ASD of ITZ was prepared with and without XDP by HME method. The ingredients of formulations 16, 17 and 18 as described in Table 13 were mixed until uniform using a mortar and pestle, and then each composition was processed by HME using a Leistritz Nano-16 co-rotating, twin-screw extruder (American Leistritz Extruder Corp., Somerville, N.J.) equipped with twin-screws containing kneading elements (30°) and without a die. Conveying, kneading, and mixing elements were used in the screw design, and each operation conditions are illustrated in Table 2 (Rp.4: 1-kneading, 50 rpm condition). A twin-screw volumetric feeder (Brabender Technology, Duisburg, Germany) set on top of the barrel feed zone provided an accurate 1 g/min feed rate of the powder blend that was mixed until uniform. The barrel configuration consisted of a feed zone, closed barrel, closed venting zone, and a closed zone before the top block. The feeding zone was maintained at room temperature conditions with water circulation. The barrel temperatures for zones 1, 2, and 3 were 160° C., 160° C., and 160° C., respectively. All extrudates were cooled to room temperature, and then milled and sieved through a 212 μm screen. However, in formulation 17, ITZ:AF4M sample's color was brown, it means the sample was scorched (see, FIG. 18) and this sample was not used at dissolution test due to not acceptable for pharmaceutical product.

TABLE 13
Formulations made by thermal processing comprising ITZ.

| | Thermal | | | | | |
|---|---|---|---|---|---|---|
| | Processing | ITZ | AF4M | AF100 | XDP | Ratio |
| Formulation | Technique | (%) | (%) | (%) | (%) | (ITZ:Polymer:XDP) |
| 16 | HME | 50 | 20 | — | 30 | 5:2:3 |
| 17 | HME | 50 | 50 | — | — | 5:5:0 |
| 18 | HME | 50 | — | 20 | 30 | 5:2:3 |

HM: Hot Melt Granulation; ITZ: Itraconazole; AF4M: Hypromellose (Affinisol HPMC HME 4M); AF100: Hypromellose (Affinisol HPMC HME 100LV); XDP: Hydrous silicon dioxide (Syloid XDP 3050)

A Hanson SR8PLUS dissolution test apparatus 2 (Hanson Research Co., Chatsworth, Calif.) (paddles) was used to perform dissolution testing. The paddle speed and temperature were set to 100 rpm and 37° C.±0.5° C., respectively. pH 6.8 phosphate buffer (900 mL) was pre-heated to 37° C. in each dissolution vessel. Aliquots of granules from formulations 16, 18 and PM (formulation 17) containing about 200 mg ITZ equivalent (n=2-3) were then added immediately to the dissolution vessel. 2 mL samples of the dissolution media were withdrawn at 5 min, 15 min, 30 min, 1 h, 2 h, 4 h and 6 h, and filtered through a 0.45-μm 25-mm PES membrane filter. A 500 μL aliquot of the filtered solution was then diluted with 500 μL HPLC grade acetonitrile, and the concentration of ITZ in the diluted sample was determined using HPLC. See FIG. 19. ITZ solubility is 0.0009 mg (=0.001 μg/mL*×900 mL/1000, *: Wlodarski et al., 2018).

### Example 12—Effect of Storage Shelf-Life on ITZ Ternary ASD Dissolution at a pH at which ITZ is Insoluble

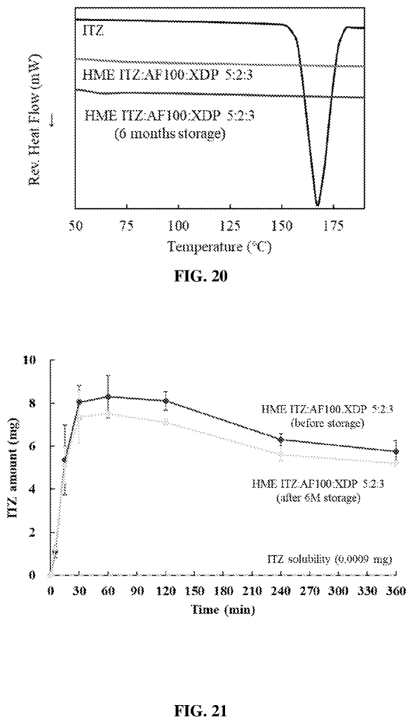

mDSC equipped with a DSC refrigerated cooling system (DSC 2920, TA Instruments, New Castle, Del.) was employed. Dry nitrogen gas at a flow rate of 40 mL/min throughout the testing was used to purge the DSC cell. Samples were accurately weighed in aluminum sample pan kits (PerkinElmer Inc., Shelton, Conn.) and crimped before analysis. Samples were heated from 25° C. to 220° C. with a heating ramp rate of 10° C./min using a 1° C./60 sec modulation program. TA Universal Analysis 2000 software was used to process the raw data. See FIG. 20.

A Hanson SR8PLUS dissolution test apparatus 2 (Hanson Research Co., Chatsworth, Calif.) (paddles) was used to perform dissolution testing. The paddle speed and temperature were set to 100 rpm and 37° C.±0.5° C., respectively. pH 6.8 phosphate buffer (900 mL) was pre-heated to 37° C. in each dissolution vessel. Aliquots of granules from formulations 18 containing about 200 mg ITZ equivalent before and after storage of 6 months in a desiccator at room temperature (n=2-3) were then added immediately to the dissolution vessel. 2 mL samples of the dissolution media were withdrawn at 5 min, 15 min, 30 min, 1 h, 2 h, 4 h and 6 h, and filtered through a 0.45-μm 25-mm PES membrane filter. A 500 μL aliquot of the filtered solution was then diluted with 500 μL HPLC grade acetonitrile, and the concentration of ITZ in the diluted sample was determined using HPLC. See FIG. 21.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 8,486,423
U.S. Pat. No. 9,339,440
Affinisol™ HPMC HME for hot melt extrusion, D.P.F. Solutions, Editor.
Altamimi and Neau, Drug Development and Industrial Pharmacy, 42(3): p. 446-455, 2016.
Aso et al., Chemical and Pharmaceutical Bulletin, 55(8): p. 1227-1231, 2007.
Bahl and Bogner, Pharmaceutical Research, 23(10): p. 2317-2325. 2006.
Bansal et al., AAPS PharmSciTech, 17(2): p. 318-327, 2016.
Bhargavi et al., The Pharma Innovation, 4(10): p. 88-92, 2015.
Brettmann et al., Journal of Pharmaceutical Sciences, 101(4): p. 1538-1545, 2012.
Brough and Williams, International Journal of Pharmaceutics, 453(1): p. 157-166, 2013.
Brouwers et al., Journal of Pharmaceutical Sciences, 98(8): p. 2549-2572. 2009.
Brown et al., “Hot-melt extrusion for solid dispersions: composition and design considerations.” In: Shah N, Sandu H, Choi D K, Chokshi H, Malick A W editors. Amorphous Solid Dispersions. Second ed. Cham, N.Y.: Springer; p. 197-230, 2014.
Crowley et al., Drug Dev. Ind. Pharm., 33(10):909-926, 2007.
Chauhan et al., Pharmaceutical Research, 31(2): p. 500-515, 2014.
Clauss et al., Acta Polymerica, 44(1): p. 1-17, 1993.
DiNunzio et al., Drug Development and Industrial Pharmacy, 36(9):1064-78, 2010d.
DiNunzio et al., European Journal of Pharmaceutical Sciences, 40(3):179-87, 2010c.
DiNunzio et al., European Journal of Pharmaceutics and Biopharmaceutics, 74(2):340-51, 2010a.
DiNunzio et al., Journal of Pharmaceutical Sciences, 99(3):1239-53, 2010b.
Ellenberger et al., AAPS PharmSciTech, 19(5): p. 1957-1970, 2018.
Fujii et al., International Journal of Pharmaceutics, 293(1): p. 145-153, 2005.
Goddeeris et al., European Journal of Pharmaceutical Sciences, 34(4): p. 293-302, 2008.
Gupta et al., Journal of Excipients and Food Chemicals, (1): p. 32-45, 2014.
Guzmán et al., Journal of Pharmaceutical Sciences, 96(10): p. 2686-2702, 2007.
Haser et al., International Journal of Pharmaceutics, 524(1-2): p. 55-64, 2017
Hoashi et al., Asian Journal of Pharmaceutical Sciences, 6(3-4): p. 101-108, 2011.
Hong et al., Drug Delivery, 23(1): p. 316-327, 2016.
Huang et al., AAPS PharmSciTech, 17(1): p. 106-119, 2016.
Huang et al., European Journal of Pharmaceutics and Biopharmaceutics, 119: p. 56-67, 2017
Hanada et al. (1), Journal of Pharmaceutical Sciences, 107(1): p. 362-371, 2018.
Hanada et al. (2), International Journal of Pharmaceutics, 548(1): p. 571-585, 2018.
Hughey et al., AAPS PharmSciTech, 11(2):760-74, 2010.
Hughey et al., International Journal of Pharmaceutics, 419(1):222-30, 2011.
Kaneniwa et al., Chemical and Pharmaceutical Bulletin, 33(8): p. 3447-3455, 1985.
Keen et al., Int. J. Pharm., 450(1-2):185-196, 2013.
LaFountaine et al., AAPS PharmSciTech, 17(1):43-, 2016a.
LaFountaine et al., European Journal of Pharmaceutics and Biopharmaceutics, 101:72-81, 2016.
Laine et al., International Journal of Pharmaceutics, 512(1): p. 118-125, 2016.
Lang et al., Drug Dev. Ind. Pharm., 40(9):1133-1155, 2014.
Lin and Huang, International Journal of Pharmaceutics, 399(1): p. 109-115, 2010.
Madieh et al., Journal of Pharmaceutical Sciences, 96(4): p. 851-863, 2007.
Maleki et al., J. Controlled Release, 262:329-347, 2017.
Maniruzzaman et al., International Journal of Pharmaceutics, 496(1): p. 42-51, 2015.
Marsac et al., Journal of Pharmaceutical Sciences, 99(1): p. 169-185, 2010.
Marsac et al., Pharmaceutical Research, 23(10): p. 2417, 2006.
Marsac et al., Pharmaceutical Research, 26(1): p. 139, 2008.
Martin, Pharmaceutical Technology, 32(10): p. 76-86, 2008
Matsumoto et al., Chemical and Pharmaceutical Bulletin, 48(2): p. 314-318, 1998.
Münzenberg and Moller, Pharmaceutical Technology, 2017(2): p. 12-15, 2017.
Nakagami, H., Chemical & Pharmaceutical Bulletin, 39(9): p. 2417-2421, 1991.
Nakahashi et al., Advanced Powder Technology, 25(2): p. 716-721, 2014.
Nakai et al., Chemical and Pharmaceutical Bulletin, 32(11): p. 4566-4571, 1984.
Nováková et al., Journal of Pharmaceutical and Biomedical Analysis, 37(5): p. 899-905, 2005.
Patel and Dave, AAPS PharmSciTech, 14(2): p. 620-628, 2013.
Prasad et al., “Emerging Technologies to Increase the Bioavailability of Poorly Water-Soluble Drugs,” In: Williams III R O, Watts A B, Miller D A, editors. Formulating Poorly Water Soluble Drugs. Second ed. Cham, Switzerland: Springer International; p. 691-740, 2016.
Purohit and Taylor, Molecular Pharmaceutics, 12(5): p. 1623-1635, 2015.
Purohit et al., Pharmaceutical Research, 34(7): p. 1364-1377, 2017.
Repka et al., Drug Dev. Ind. Pharm., 33(10):1043-1057, 2007.
Salonen, et al., Journal of Controlled Release, 108(2): p. 362-374, 2005.
Sayed et al., AAPS PharmSciTech, 18(5):1507-1525, 2017.
Shibata et al., International Journal of Pharmaceutics, 365(1): p. 53-60, 2009.
Stejskal et al., Macromolecules, 14(2): p. 275-279, 1981.
Suhrenbrock et al., International Journal of Pharmaceutics, 412(1): p. 28-36, 2011.
Takeuchi et al., International Journal of Pharmaceutics, 293(1): p. 155-164, 2005.
Tanabe et al., International Journal of Pharmaceutics, 429(1): p. 38-45, 2012.
Tian et al., Molecular Pharmaceutics, 10(1): p. 236-248, 2013.
Ueda et al., Journal of Pharmaceutical Sciences, 103(9): p. 2829-2838, 2014.
Ukmar et al., The Journal of Physical Chemistry C, 116(4): p. 2662-2671, 2012.
Van Speybroeck et al., European Journal of Pharmaceutics and Biopharmaceutics, 75(3): p. 354-365, 2010. Vynckier et al., J. Pharm. Pharmacol., 66(2):167-179, 2014.
Vogt et al., Pharmaceutical Research, 30(9): p. 2315-2331, 2013.
Watanabe et al., International Journal of Pharmaceutics, 226(1): p. 81-91, 2001.
Watanabe et al., International Journal of Pharmaceutics, 250(1): p. 283-286, 2003.
Watanabe et al., Journal of Solid State Chemistry, 164(1): p. 27-33, 2002.
Wlodarski et al., Pharmaceutical Research, 35(1): 16
Wu et al., Polymer, 43(1): p. 171-176, 2002.
Xiang and Anderson, Journal of Pharmaceutical Sciences, 102(3): p. 876-891, 2013.
Yang et al., Molecular Pharmaceutics, 13(10): p. 3494-3505, 2016.
Yani et al., Industrial & Engineering Chemistry Research, 56(44): p. 12698-12707, 2017.
Yoshioka et al., Journal of Pharmaceutical Sciences, 83(12): p. 1700-1705, 1994.
Yuan et al., Molecular Pharmaceutics, 11(1): p. 329-337, 2014.
Zhao et al., Journal of Pharmaceutical Sciences, 100(8): p. 3196-3207, 2011.

1. A pharmaceutical composition comprising:
(A) a therapeutic agent, wherein the therapeutic agent comprises at least about 50% w/w of the pharmaceutical composition;
(B) one or more pharmaceutically acceptable polymers; and
(C) a non-preloaded mesoporous carrier.

2. The pharmaceutical composition of claim 1, wherein the pharmaceutical composition has been processed through a thermal process or a fusion-based high energy mixing process that does not require external heat input.

3.-10. (canceled)

11. The pharmaceutical composition of claim 1, wherein the pharmaceutical composition is substantially free of a solvent.

12.-14. (canceled)

15. The pharmaceutical composition of claim 1, wherein the therapeutic agent has a solubility in water of less than about 5 mg/mL.

16.-18. (canceled)

19. The pharmaceutical composition of claim 1, wherein the therapeutic agent is substantially present as an amorphous form or as a molecular solution.

20.-28. (canceled)

29. The pharmaceutical composition of claim 1, wherein the mesoporous carrier is a silica carrier, an alumina carrier, a mixed alumino-silicate carrier, a mixed inorganic oxide carrier, a calcium carbonate carrier, or a clay carrier.

30.-34. (canceled)

35. The pharmaceutical composition of claim 1, wherein the mesoporous carrier has not been loaded with the therapeutic agent before the formulation with the pharmaceutically acceptable polymer.

36. (canceled)

37. The pharmaceutical composition of claim 1, wherein the pharmaceutically acceptable polymer and the therapeutic agent form a mixture having a Flory-Huggins interaction parameter (χ) of greater than 0.25 as determined by differential scanning calorimetry (DSC).

38.-39. (canceled)

40. The pharmaceutical composition of claim 1, wherein the pharmaceutical composition has a specific surface area of greater than about 5 m²/g as measured by BET.

41.-43. (canceled)

44. The pharmaceutical composition of claim 1, wherein the pharmaceutical composition comprises from about 50% w/w to about 98% w/w therapeutic agent relative to the total weight of the pharmaceutical composition.

45.-46. (canceled)

47. The pharmaceutical composition of claim 1, wherein the pharmaceutical composition comprises from about 1% w/w to about 49% w/w mesoporous carrier relative to the total weight of the pharmaceutical composition.

48.-50. (canceled)

51. The pharmaceutical composition of claim 1, wherein the pharmaceutical composition comprises from about 1% w/w to about 49% w/w pharmaceutically acceptable polymer relative to the total weight of the pharmaceutical composition.

52.-60. (canceled)

61. A solvent free method of preparing a pharmaceutical composition comprising:
(A) obtaining a composition comprising:
(1) a therapeutic agent, wherein the therapeutic agent comprises at least about 50% w/w of the composition;
(2) a mesoporous carrier; and
(3) one or more pharmaceutically acceptable polymers;
(B) heating the composition through a thermal process or a fusion-based high energy mixing process to form a pharmaceutical composition.

62. The method of claim 61, wherein the composition is obtained by adding the therapeutic agent, the mesoporous carrier, and the pharmaceutically acceptable polymers.

63.-64. (canceled)

65. The method of claim 61, wherein the thermal process is hot melt extrusion.

66.-68. (canceled)

69. The method of claim 61, wherein the fusion-based high energy mixing process that does not require external heat input which results in an increase in temperature.

70.-71. (canceled)

72. The method of claim 61, wherein the pharmaceutically acceptable polymers and the therapeutic agent form a mixture have a Flory-Huggins interaction parameter (χ) of greater than 0.25 as determined by differential scanning calorimetry (DSC).

73.-108. (canceled)

109. The method of claim 61, wherein the mesoporous carrier is a silica carrier, an alumina carrier, a mixed alumino-silicate carrier, a mixed inorganic oxide carrier, a calcium carbonate carrier, or a clay carrier.

110.-113. (canceled)

114. A pharmaceutical composition prepared according to the methods of claim 61.

115.-117. (canceled)

118. A method of treating a disease or disorder in a patient in need thereof comprising administering to the patient a therapeutically effective amount of a pharmaceutical composition of claim 1 comprising a therapeutic agent effective to treat the disease or disorder.

119. (canceled)
